(19) United States
(12) Patent Application Publication
(10) Pub. No.: US 2020/0319197 A1
(43) Pub. Date: Oct. 8, 2020

(54) Biomarkers of Preterm Birth

(71) Applicant: Carmentix PTE. Ltd., Singapore (SG)

(72) Inventors: Nir Arbel, Singapore (SG); San Min Leow, Singapore (SG); Xiang Qian Lin, Singapore (SG); Eitan Rubin, Singapore (SG)

(21) Appl. No.: 16/758,297

(22) PCT Filed: Oct. 30, 2018
(86) PCT No.: PCT/EP2018/079639

§ 371 Date: Apr. 22, 2020

(30) Foreign Application Priority Data
Oct. 30, 2017 (SG) 10201708890Y

(51) Int. Cl. G01N 33/68; A61K 31/57; G01N 33/543

(52) U.S. Cl. CPC G01N 33/689; A61K 31/57; A61K 45/06; G01N 2800/50; G01N 2800/368; G01N 33/543

(57) ABSTRACT

The present invention relates to biomarkers, and particularly, although not exclusively, to biomarkers of preterm birth. The biomarkers are useful, several weeks or months prior to birth, for distinguishing individuals at risk of experiencing birth before 37 weeks of gestation. The biomarkers, namely ECM1, FGA, EFEMP1, GGH, PEDF, PTN and LAMC2, are identified by a proteomics analysis, as being differentially expressed in vaginal fluid by women going to have preterm birth.

This application claims priority from SG10201708890Y filed 30 Oct. 2017, the contents and elements of which are herein incorporated by reference for all purposes.

### FIELD OF THE INVENTION

The present invention relates to biomarkers, and particularly, although not exclusively, to biomarkers of preterm birth. The biomarkers are useful, several weeks or months prior to birth, for distinguishing individuals at risk of experiencing birth before 37 weeks of gestation.

### BACKGROUND

Preterm birth is defined by birth that takes place before the completion of 37 weeks of gestation. It is estimated that over 15 million babies are born preterm annually. Globally, preterm birth is one of the leading causes of death for children under the age of five with an estimated of one million preterm birth-related mortalities. Many of the survivors face a lifetime of challenging disabilities which include learning disabilities and visual and hearing problems. Although neonatology advances in the past decades has increased survival rates for preterm birth, above 20% of preterm neonates will suffer at least one major disability including chronic lung disease, impaired mental development, cerebral palsy, deafness, or blindness.

There is a significant need to identify pregnant women who are at risk of preterm birth. In the current paradigms, treatment for high-risk pregnancies involves prophylactic treatment or enhanced surveillance or close monitoring of the pregnancy, which reduces preterm birth rates. However, in most cases, classification of pregnancies as high-risk is attributed to prior medical history or clinical examinations, identifying only a small subsection of the true high-risk pregnancies prone to preterm birth. Still, the majority of pregnancies that are prone to preterm births are not identified at early stages and hence early medical intervention for such cases is not possible.

There are several tests in the market for risk assessment of preterm birth for women presenting risk symptoms. One such example is the Fetal Fibronectin (fFN) test which provides a risk assessment for symptomatic women. Fetal fibronectin is present in the vagina if a preterm delivery is likely to occur; hence the fFN test is commonly used in pregnant women with symptoms indicating a possibility for preterm birth, such as contractions, vaginal bleeding, fluid leaking from the vagina, increased vaginal discharge, backache and cramp in lower abdomen. The strength of the fFN test lies in its high negative predictive value for up to 10 days following the test (i.e. A negative result means that there is a low possibility of preterm labour within the next 7 to 10 days following the test). However, when the fFN test is positive, the results are less conclusive.

Patient management varies based on risk factors. Prophylactic treatment such as progesterone has been shown to reduce preterm birth rates in numerous clinical studies profiling women with short cervical length or prior history of preterm birth as a high-risk population. Symptomatic women may receive treatments such as tocolytics or steroids based on the risk factors. The limitation of current clinical practice is that the correlation of treatment and outcomes is very low.

Thus, there is an urgent need for effective identification of pregnancies with high-risk of preterm birth, so that appropriate treatment can be administered promptly to reduce preterm birth rates. The present invention provides biomarkers and methods for predicting risk of preterm birth to overcome at least in part some of the disadvantages. In particular, the present invention seeks to provide a risk assessment for classification of women with high-risk for preterm birth several weeks or even months before symptoms of preterm birth appear.

The present invention has been devised in light of the above considerations.

### SUMMARY OF THE INVENTION

The present invention provides novel biomarkers and methods for predicting risk or likelihood of preterm birth in a subject. The biomarkers disclosed herein were identified in a meta-data analysis, and subsequently demonstrated in patient-derived samples. Biomarkers disclosed herein differentiate samples from term and preterm individuals, weeks or months before the individual is symptomatic. Such biomarkers may be useful for identifying an individual at risk of preterm birth, and thus may be useful for guiding clinical decisions such as the initiation of treatment to prolong gestation and/or prevent or reduce the risk of preterm birth.

Methods disclosed herein can be used to determine the risk or likelihood of preterm birth in asymptomatic or symptomatic individuals. In particular embodiments the individual is asymptomatic.

As disclosed herein, ECM1, FGA, EFEMP1, GGH, PEDF, PTN and LAMC2 are biomarkers of preterm birth. Each of ECM1, FGA, EFEMP1, GGH, PEDF, PTN and LAMC2 may be used in methods for identifying individuals at risk of preterm birth, and methods for determining whether an individual is at risk of preterm birth, or for predicting whether an individual is at risk of preterm birth. Variation of the level of the biomarker, as compared to a control or reference level, may indicate that the individual is at risk of preterm birth. Such methods involve determining the level of ECM1, FGA, EFEMP1, GGH, PEDF, PTN and LAMC2 in a sample obtained from the individual being tested. In some aspects, the methods involve determining the level of all of the biomarkers ECM1, FGA, EFEMP1, GGH, PEDF, PTN and LAMC2, and predicting the risk of preterm birth.

Under-expression of certain biomarkers may indicate that the individual is at risk of preterm birth, including ECM1, GGH, LAMC2, EFEMP1, PTN and FGA.

Over-expression of certain biomarkers may indicate that the individual is at risk of preterm birth, including GGH, LAMC2, EFEMP1, PTN, FGA and PEDF.

In some cases, the biomarker may indicate that the individual is at risk of preterm birth if it is over-expressed at a certain point of gestation, or under-expressed at a different point of gestation.

Provided herein is a method for predicting whether an individual is at risk of preterm birth, the method comprising determining the level of a biomarker in a sample obtained from the individual, and classifying the individual as at risk of preterm birth or not at risk of preterm birth, based on the biomarker value, wherein the biomarker is selected from ECM1, FGA, EFEMP1, GGH, PEDF, PTN and LAMC2. The method may further comprise administering a treatment to an individual determined to be at risk. The treatment may comprise cervical cerclage or administration of one or more agents selected from a progesterone or an analogue thereof, a tocolytic, a corticosteroid, an antibiotic, an NSAID or an Omega 3 fatty acid or derivative thereof. The progesterone may be a synthetic progesterone, such as 17-a-hydroxyprogesterone caproate. The tocolytic may be magnesium sulfate, indomethacin or Nifedipine. The antibiotic may be erythromycin or penicillin. The NSAID may be indomethacin. The Omega 3 fatty acid derivative may be docosahexaenoic acid (DHA).

Also disclosed herein is a method of determining the likelihood of an individual experiencing a preterm birth, the method comprising detecting, in a sample from the individual, a biomarker value for a biomarker selected from ECM1, FGA, EFEMP1, GGH, PEDF, PTN and LAMC2, and determining the percentage likelihood that an individual will experience a preterm birth, based on the biomarker value.

Also disclosed herein is a computer implemented method for predicting whether an individual is at risk of preterm birth, the method comprising retrieving on a computer biomarker information for an individual, wherein the biomarker information comprises biomarker values corresponding to at least one biomarker selected from ECM1, FGA, EFEMP1, GGH, PEDF, PTN and LAMC2, and with the computer a classification of the biomarker value; and indicating a likelihood that the individual is at risk of preterm birth, based on the classification.

Also disclosed herein is a method of treatment comprising administration of one or more agents selected from a progesterone or an analogue thereof, a tocolytic, a corticosteroid, an antibiotic, an NSAID or an Omega 3 fatty acid or derivative thereof to an individual determined to be at risk of preterm birth based on a biomarker value for a biomarker selected from ECM1, FGA, EFEMP1, GGH, PEDF, PTN and LAMC2.

Progesterone for use in such a method is also disclosed, along with the use of progesterone in the manufacture of a medicament for use in such a method. The progesterone may be a synthetic progesterone, such as 17-a-hydroxyprogesterone caproate. The tocolytic may be magnesium sulfate, indomethacin or Nifedipine. The antibiotic may be erythromycin or penicillin. The NSAID may be indomethacin. The Omega 3 fatty acid derivative may be docosahexaenoic acid (DHA)

Also disclosed herein is a method of treatment comprising cervical cerclage to an individual determined to be at risk of preterm birth based on a biomarker value for a biomarker selected from ECM1, FGA, EFEMP1, GGH, PEDF, PTN and LAMC2. Methods for selecting an individual for treatment with cervical cerclage are also disclosed.

Also disclosed herein is a tocolytic or steroid for use in a method of treating an individual predicted to be at risk of preterm birth, wherein the individual has been determined to be at risk of preterm birth based on a biomarker value for a biomarker selected from ECM1, FGA, EFEMP1, GGH, PEDF, PTN and LAMC2. A method of treatment comprising administering a tocolytic or steroid to an individual determined to be at risk of preterm birth, wherein the individual has been determined to be at risk of preterm birth based on a biomarker value for a biomarker selected from ECM1, FGA, EFEMP1, GGH, PEDF, PTN and LAMC2 is also disclosed.

Relatedly, there is provided the use of a tocolytic or steroid for the manufacture of a medicament for the treatment of an individual determined to be at risk of preterm birth, wherein the individual has been predicted to be at risk of preterm birth based on a biomarker value for a biomarker selected from ECM1, FGA, EFEMP1, GGH, PEDF, PTN and LAMC2.

The tocolytic or steroid for use, or the medicament may be for use in a method involving determining a biomarker value for a biomarker selected from ECM1, FGA, EFEMP1, GGH, PEDF, PTN and LAMC2, and determining the risk of preterm birth based on the that biomarker value.

Also disclosed herein is one or more agents selected from a progesterone or an analogue thereof, a tocolytic, a corticosteroid, an antibiotic, an NSAID or an Omega 3 fatty acid or derivative thereof for use in a method of treating an individual predicted to be at risk of preterm birth, wherein the individual has been determined to be at risk of preterm birth based on a biomarker value for a biomarker selected from ECM1, FGA, EFEMP1, GGH, PEDF, PTN and LAMC2. The progesterone may be a synthetic progesterone, such as 17-a-hydroxyprogesterone caproate. The tocolytic may be magnesium sulfate, indomethacin or Nifedipine. The antibiotic may be erythromycin or penicillin. The NSAID may be indomethacin. The Omega 3 fatty acid derivative may be docosahexaenoic acid (DHA).

A method of treatment comprising administering one or more agents selected from a progesterone or an analogue thereof, a tocolytic, a corticosteroid, an antibiotic, an NSAID or an Omega 3 fatty acid or derivative thereof, to an individual determined to be at risk of preterm birth, wherein the individual has been determined to be at risk of preterm birth based on a biomarker value for a biomarker selected from ECM1, FGA, EFEMP1, GGH, PEDF, PTN and LAMC2 is also disclosed. The progesterone may be a synthetic progesterone, such as 17-a-hydroxyprogesterone caproate. The tocolytic may be magnesium sulfate, indomethacin or Nifedipine. The antibiotic may be erythromycin or penicillin. The NSAID may be indomethacin. The Omega 3 fatty acid derivative may be docosahexaenoic acid (DHA).

There is also provided the use of one or more agents selected from a progesterone or an analogue thereof, a tocolytic, a corticosteroid, an antibiotic, an NSAID or an Omega 3 fatty acid or derivative thereof in the manufacture of a medicament for the treatment of an individual determined to be at risk of preterm birth, wherein the individual has been determined to be at risk of preterm birth based on a biomarker value for a biomarker selected from ECM1, FGA, EFEMP1, GGH, PEDF, PTN and LAMC2. The progesterone may be a synthetic progesterone, such as 17-a-hydroxyprogesterone caproate. The tocolytic may be magnesium sulfate, indomethacin or Nifedipine. The antibiotic may be erythromycin or penicillin. The NSAID may be indomethacin. The Omega 3 fatty acid derivative may be docosahexaenoic acid (DHA).

The agent for use, or the medicament may be for use in a method involving determining a biomarker value for a biomarker selected from ECM1, FGA, EFEMP1, GGH, PEDF, PTN and LAMC2, and determining the risk of preterm birth based on the that biomarker value.

Provided herein is a method for detecting ECM1, FGA, EFEMP1, GGH, PEDF, PTN or LAMC2, the method comprising:
a. obtaining a sample of vaginal fluid from an individual;
b. detecting whether ECM1, FGA, EFEMP1, GGH, PEDF, PTN or LAMC2 is present in the vaginal fluid sample by contacting the vaginal fluid sample with an anti-ECM1, anti-FGA, anti-EFEMP1, anti-GGH, anti-PEDF, anti-PTN or anti-LAMC2 antibody and detecting binding between ECM1, FGA, EFEMP1, GGH, PEDF, PTN or LAMC2 and the antibody.

Also provided herein is a method for determining that an individual is at risk of preterm birth, said method comprising:
a. obtaining a sample from an individual;
b. detecting whether ECM1, FGA, EFEMP1, GGH, PEDF, PTN or LAMC2 is present in the sample by contacting the sample with an anti-ECM1, anti-FGA, anti-EFEMP1, anti-GGH, anti-PEDF, anti-PTN or anti-LAMC2 antibody and detecting binding between ECM1, FGA, EFEMP1, GGH, PEDF, PTN or LAMC2 and the antibody; and
c. determining that the individual is at risk of preterm birth when the presence of ECM1, FGA, EFEMP1, GGH, PEDF, PTN or LAMC2 in the vaginal fluid sample is detected.

In some cases, the antibody is derived from, mouse, rabbit or goat, preferably, mouse or rabbit. The antibody may be human, humanised or chimeric.

Preferably, the sample is a vaginal fluid sample. The vaginal fluid sample may be a cervicovaginal fluid sample. Alternatively, the sample is an amniotic fluid sample.

A method of determining that an individual is at risk of preterm birth and prolonging gestation in that individual, the method comprising:
a. obtaining a sample from an individual;
b. detecting whether ECM1, FGA, EFEMP1, GGH, PEDF, PTN or LAMC2 is present in the plasma sample;
c. determining that the individual is at risk of preterm birth when the presence of ECM1, FGA, EFEMP1, GGH, PEDF, PTN or LAMC2 in the vaginal fluid sample is detected; and
d. administering an effective amount of one or more agents selected from a progesterone or an analogue thereof, a tocolytic, a corticosteroid, an antibiotic, an NSAID or an Omega 3 fatty acid or derivative thereof. The progesterone may be a synthetic progesterone, such as 17-a-hydroxyprogesterone caproate. The tocolytic may be magnesium sulfate, indomethacin or Nifedipine. The antibiotic may be erythromycin or penicillin. The NSAID may be indomethacin. The Omega 3 fatty acid derivative may be docosahexaenoic acid (DHA) to the individual determined to be at risk of preterm birth or selecting the individual for treatment with an effective amount of tocolytic or steroid, if the individual is determined to be at risk of preterm birth.

In one aspect, there is disclosed a kit for use in predicting the risk or likelihood of preterm birth in a subject, the kit comprising anti-ECM1, anti-FGA, anti-EFEMP1, anti-GGH, anti-PEDF, anti-PTN or anti-LAMC2 antibody. Preferably, the anti-ECM1, anti-FGA, anti-EFEMP1, anti-GGH, anti-PEDF, anti-PTN or anti-LAMC2 antibody is capable of selectively binding to ECM1, FGA, EFEMP1, GGH, PEDF, PTN or LAMC2.

Certain aspects disclosed herein describe computer-implemented methods for determining the risk of preterm birth in an individual. The methods may involve providing data corresponding to the level of at least one of ECM1, FGA, EFEMP1, GGH, PEDF, PTN or LAMC2 in a sample obtained from the individual; performing, with the computer, a classification of the biomarker value; and determining the risk of preterm birth in the individual, based on the classification.

The invention includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying figures in which:

FIG. 1. This figure represents the difference in ECM1 level between term and preterm samples. The mean biomarker protein concentration was calculated based on the outcomes of an ELISA immunoassay adjusted for total protein concentration. Term (n=136) and Preterm (n=64) samples collected between 19-37 weeks of gestation and the mean difference between the entire groups is represented. Data were analysed using Student's t-test. P=p-value.

FIG. 2. This figure represents the difference in ECM1 level between term and preterm samples based on the gestational age at the time of sampling. Samples were grouped into four groups based on the sampling time, 18+0-23+6 days Weeks of gestation (18-24), 24+0-27+6 days weeks of gestation (24-28), 28+0-31+6 days weeks of gestation (28-32) and 32+0-37+6 days weeks of gestation (32-37). The groups were then divided into term and preterm samples and the mean was calculated and is represented in the bar graph.

FIG. 3. The figure represents the difference in ECM1 level between term and preterm birth women based on the time from sample collection to delivery. The samples were grouped into 4 groups of 4-week interval to assess the difference between term and preterm samples based on the weeks before birth. The groups are more than 12 weeks between sampling and delivery (>12), 9-12, 5-8 and 1-4 weeks from sampling to delivery (9-12, 5-8, 1-4), respectively. The mean was calculated for each group.

FIG. 4. This figure represents the difference in GGH level between term and preterm samples. The mean biomarker protein concentration was calculated based on the outcomes of an ELISA immunoassay adjusted for total protein concentration. Term (n=136) and Preterm (n=64) samples collected between 19-37 weeks of gestation and the mean difference between the entire groups is represented. Data were analysed using Student's t-test. P=p-value.

FIG. 5. This figure represents the difference in GGH level between term and preterm samples based on the gestational age at the time of sampling. Samples were grouped into four groups based on the sampling time, 18+0-23+6 days Weeks of gestation (18-24), 24+0-27+6 days weeks of gestation (24-28), 28+0-31+6 days weeks of gestation (28-32) and 32+0-37+6 days weeks of gestation (32-37). The groups were then divided into term and preterm samples and the mean was calculated and is represented in the bar graph.

FIG. 6. The figure represents the difference in GGH level between term and preterm birth women based on the time from sample collection to delivery. The samples were grouped into 4 groups of 4-week interval to assess the difference between term and preterm samples based on the weeks before birth. The groups are more than 12 weeks between sampling and delivery (>12), 9-12, 5-8 and 1-4 weeks from sampling to delivery (9-12, 5-8, 1-4), respectively. The mean was calculated for each group.

FIG. 7. This figure represents the difference in LAMC2 level between term and preterm samples. The mean biomarker protein concentration was calculated based on the outcomes of an ELISA immunoassay adjusted for total protein concentration. Term (n=136) and Preterm (n=64) samples collected between 19-37 weeks of gestation and the mean difference between the entire groups is represented. Data were analysed using Student's t-test. P=p-value.

FIG. 8. This figure represents the difference in LAMC2 level between term and preterm samples based on the gestational age at the time of sampling. Samples were grouped into four groups based on the sampling time, 18+0-23+6 days Weeks of gestation (18-24), 24+0-27+6 days weeks of gestation (24-28), 28+0-31+6 days weeks of gestation (28-32) and 32+0-37+6 weeks of gestation (32-37). The groups were then divided into term and preterm samples and the mean was calculated and is represented in the bar graph.

FIG. 9. The figure represents the difference in LAMC2 level between term and preterm birth women based on the time from sample collection to delivery. The samples were grouped into 4 groups of 4-week interval to assess the difference between term and preterm samples based on the weeks before birth. The groups are more than 12 weeks between sampling and delivery (>12), 9-12, 5-8 and 1-4 weeks from sampling to delivery (9-12, 5-8, 1-4), respectively. The mean was calculated for each group.

FIG. 10. This figure represents the difference in EFEMP1 level between term and preterm samples. The mean biomarker protein concentration was calculated based on the outcomes of an ELISA immunoassay adjusted for total protein. Term (n=136) and Preterm (n=64) samples collected between 19-37 weeks of gestation and the mean difference between the entire groups is represented. Data were analysed using Student's t-test. P=p-value.

FIG. 11. This figure represents the difference in EFEMP1 level between term and preterm samples based on the gestational age at the time of sampling. Samples were grouped into four groups based on the sampling time, 18+0-23+6 days Weeks of gestation (18-24), 24+0-27+6 days weeks of gestation (24-28), 28+0-31+6 days weeks of gestation (28-32) and 32+0-37+6 days weeks of gestation (32-37). The groups were then divided into term and preterm samples and the mean was calculated and is represented in the bar graph.

FIG. 12. The figure represents the difference in EFEMP1 level between term and preterm birth women based on the time from sample collection to delivery. The samples were grouped into 4 groups of 4-week interval to assess the difference between term and preterm samples based on the weeks before birth. The groups are more than 12 weeks between sampling and delivery (>12), 9-12, 5-8 and 1-4 weeks from sampling to delivery (9-12, 5-8, 1-4), respectively. The mean was calculated for each group.

FIG. 13. This figure represents the difference in PTN level between term and preterm samples. The mean biomarker protein concentration was calculated based on the outcomes of an ELISA immunoassay adjusted for total protein concentration. Term (n=136) and Preterm (n=64) samples collected between 19-37 weeks of gestation and the mean difference between the entire groups is represented. Data were analysed using Student's t-test. P=p-value.

FIG. 14. This figure represents the difference in PTN level between term and preterm samples based on the gestational age at the time of sampling. Samples were grouped into four groups based on the sampling time, 18+0-23+6 days Weeks of gestation (18-24), 24+0-27+6 days weeks of gestation (24-28), 28+0-31+6 days weeks of gestation (28-32) and 32+0-37+6 days weeks of gestation (32-37). The groups were then divided into term and preterm samples and the mean was calculated and is represented in the bar graph.

FIG. 15. The figure represents the difference in PTN level between term and preterm birth women based on the time from sample collection to delivery. The samples were grouped into 4 groups of 4 week interval to assess the difference between term and preterm samples based on the weeks before birth. The groups are more than 12 weeks between sampling and delivery (>12), 9-12, 5-8 and 1-4 weeks from sampling to delivery (9-12, 5-8, 1-4), respectively. The mean was calculated for each group.

FIG. 16. This figure represents the difference in FGA level between term and preterm samples. The mean biomarker protein concentration was calculated based on the outcomes of an ELISA immunoassay adjusted for total protein. Term (n=136) and Preterm (n=64) samples collected between 19-37 weeks of gestation and the mean difference between the entire groups is represented. Data were analysed using Student's t-test. P=p-value.

FIG. 17. This figure represents the difference in FGA level between term and preterm samples based on the gestational age at the time of sampling. Samples were grouped into four groups based on the sampling time, 18+0-23+6 days Weeks of gestation (18-24), 24+0-27+6 days weeks of gestation (24-28), 28+0-31+6 days weeks of gestation (28-32) and 32+0-37+6 days weeks of gestation (32-37). The groups were then divided into term and preterm samples and the mean was calculated and is represented in the bar graph.

FIG. 18. The figure represents the difference in FGA level between term and preterm birth women based on the time from sample collection to delivery. The samples were grouped into 4 groups of 4-week interval to assess the difference between term and preterm samples based on the weeks before birth. The groups are more than 12 weeks between sampling and delivery (>12), 9-12, 5-8 and 1-4 weeks from sampling to delivery (9-12, 5-8, 1-4), respectively. The mean was calculated for each group.

FIG. 19. This figure represents the difference in PEDF level between term and preterm samples. The mean biomarker protein concentration was calculated based on the outcomes of an ELISA immunoassay adjusted for total protein. Term (n=136) and Preterm (n=64) samples collected between 19-37 weeks of gestation and the mean difference between the entire groups is represented. Data were analysed using Student's t-test. P=p-value.

FIG. 20. This figure represents the difference in PEDF level between term and preterm samples based on the gestational age at the time of sampling. Samples were grouped into four groups based on the sampling time, 18+0-23+6 days Weeks of gestation (18-24), 24+0-27+6 days weeks of gestation (24-28), 28+0-31+6 days weeks of gestation (28-32) and 32+0-37+6 days weeks of gestation (32-37). The groups were then divided into term and preterm samples and the mean was calculated and is represented in the bar graph.

FIG. 21. The figure represents the difference in PEDF level between term and preterm birth women based on the time from sample collection to delivery. The samples were grouped into 4 groups of 4-week interval to assess the difference between term and preterm samples based on the weeks before birth. The groups are more than 12 weeks between sampling and delivery (>12), 9-12, 5-8 and 1-4 weeks from sampling to delivery (9-12, 5-8, 1-4), respectively. The average mean was calculated for each group.

FIG. 22, Effect of H2O2 on cell viability and proliferation. Ect1 and End1 cells were treated with 50 μM, 100 μM, 200 μM and 400 μM H2O2 for 24 h. Cell viability and proliferation of Ect1 and End1 was assessed by MTT assay, and were shown as % of untreated control (0 μM H2O2). The data are represented as the mean±SEM and were analyzed by Student's t-test. *P<0.05 compared to respective controls, n=3.

FIG. 23 Effect of H2O2 on ECM1 expression of Ect1 and End1 cells. ECM1 in medium of (a) Ect1 and (b) End1 post-H2O2 treatment (24 h) was quantified by ELISA. Fold change in ECM1 expression upon H2O2 treatment was calculated by dividing normalized ECM1 level (pg/mg total protein) in treated cells to untreated cells (control). The data are represented as the mean±SEM from at least 4 independent experiments and were analyzed by Student's t-test. *P<0.05 compared to the control.

FIG. 24. Effect of H2O2 on LAMC2 expression of Ect1 and End1 cells. LAMC2 in medium of (a) Ect1 and (b) End1 post-H2O2 treatment (24 h) was quantified by ELISA. Fold change in LAMC2 expression upon H2O2 treatment was calculated by dividing normalized LAMC2 level (pg/mg total protein) in treated cells to untreated cells (control). The data are represented as the mean±SEM from at least 4 independent experiments and were analyzed by Student's t-test. *P<0.05 compared to the control.

FIG. 25. Effect of H2O2 on FGA expression of Ect1 and End1 cells. FGA in medium of (a) Ect1 and (b) End1 post-H2O2 treatment (24 h) was quantified by ELISA. Fold change in FGA expression upon H2O2 treatment was calculated by dividing normalized FGA level (ng/mg total protein) in treated cells to untreated cells (control). The data are represented as the mean±SEM from at least 5 independent experiments and were analyzed by Student's t-test. **P<0.005 compared to the control.

FIG. 26. Effect of H2O2 on GGH expression of Ect1 and End1 cells. GGH in medium of (a) Ect1 and (b) End1 post-H2O2 treatment (24 h) was quantified by ELISA. Fold change in GGH expression upon H2O2 treatment was calculated by dividing normalized GGH level (ng/mg total protein) in treated cells to untreated cells (control). The data are represented as the mean±SEM from at least 5 independent experiments and were analyzed by Student's t-test.

FIG. 27. Effect of LPS on ECM1 expression of Ect1 and End1 cells. ECM1 in medium of Ect1 and End1 post-LPS treatment (24 h) was quantified by ELISA. Fold change in ECM1 expression upon LPS treatment was calculated by dividing normalized ECM1 level (pg/mg total protein) in treated cells to untreated cells (control). The data are represented as the mean±SEM and were analyzed by Student's t-test, n=5. *P<0.05, to respective control.

FIG. 28. Effect of LPS on LAMC2 expression of Ect1 and End1 cells. LAMC2 in medium of Ect1 and End1 post-LPS treatment (24 h) was quantified by ELISA. Fold change in LAMC2 expression upon LPS treatment was calculated by dividing normalized LAMC2 level (pg/mg total protein) in treated cells to untreated cells (control). The data are represented as the mean±SEM and were analyzed by Student's t-test, n=5. *P<0.05, **P<0.05, to respective control.

FIG. 29. Effect of LPS on GGH expression of Ect1 and End1 cells. GGH in medium of Ect1 and End1 post-LPS treatment (24 h) was quantified by ELISA. Fold change in GGH expression upon LPS treatment was calculated by dividing normalized GGH level (ng/mg total protein) in treated cells to untreated cells (control). The data are represented as the mean±SEM and were analyzed by Student's t-test, n=5.

FIG. 30. Effect of LPS on FGA expression of Ect1 and End1 cells. FGA in medium of Ect1 and End1 post-LPS treatment (24 h) was quantified by ELISA. Fold change in FGA expression upon LPS treatment was calculated by dividing normalized FGA level (ng/mg total protein) in treated cells to untreated cells (control). The data are represented as the mean±SEM and were analyzed by Student's t-test, n=5.

FIG. 31. Effect of H2O2 on EFEMP1 expression of Ect1 cells. EFEMP1 in medium of Ect1 post-H2O2 treatment (24 h) was quantified by ELISA. Fold change in EFEMP1 expression upon H2O2 treatment was calculated by dividing normalized EFEMP1 level (ng/mg total protein) in treated cells to untreated cells (control). The data are represented as the mean±SEM from at least 5 independent experiments and were analyzed by Student's t-test. **P<0.05 compared to the control.

FIG. 32. Effect of H2O2 on EFEMP1 expression of End1 cells. EFEP1 in medium of End1 post-H2O2 treatment (24 h) was quantified by ELISA. Fold change in EFEMP1 expression upon H2O2 treatment was calculated by dividing normalized EFEMP1 level (ng/mg total protein) in treated cells to untreated cells (control). The data are represented as the mean±SEM from at least 5 independent experiments and were analyzed by Student's t-test. **P<0.005 compared to the control.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

Methods and biomarkers described herein may be useful for determining whether an individual is at risk of preterm birth, identifying an individual at risk of preterm birth, or predicting the risk of preterm birth in an individual.

The term “predicting” is used interchangeably with “determining” herein, and is used to say or estimate that preterm birth will happen in an individual. Methods disclosed herein may be used to determine or predict the likelihood (or “risk”) that an individual will experience preterm birth.

Preterm birth is birth that occurs before the mother has reached 37 weeks of gestation. Preterm birth is subdivided late preterm birth 35+0 days to 36+6 days weeks of gestation, moderate preterm birth 32+0 days to 34+6 days of gestation and early preterm is prior to 32 weeks of gestation.

The cause of preterm birth is often not known. Risk factors include diabetes, high blood pressure, being pregnant with more than one baby, being either obese or underweight, a number of vaginal infections, tobacco smoking, and psychological stress, among others.

Preeclampsia is clinically indicated as hypertension and proteinuria manifesting between 20 weeks of gestation and up 6 weeks post-partum. Whilst preeclampsia can lead to preterm birth, in many cases it does not. Preeclampsia is just one factor that may contribute to an increased risk of preterm birth, and thus factors that are known to cause or be associated with preeclampsia are not necessarily causative or associated with preterm birth.

### Biomarkers

The methods disclosed herein involve the determination of the presence or absence of, or quantification of the level of a biomarker selected from the group consisting of ECM1, FGA, EFEMP1, GGH, PEDF, PTN and LAMC2.

Each of the biomarkers used herein may be used alone or in combination with other biomarkers of preterm birth. In some assays, some or all of the biomarker proteins disclosed herein are used in combination. Moreover, the biomarkers may be used together with one or more other indicators of preterm birth, including the Fetal Fibronectin (fFN) test, contractions, vaginal bleeding, fluid leaking from the vagina, increased vaginal discharge, backache and cramping in lower abdomen.

### Extracellular Matrix Protein 1 (ECM1); UniProtKB-Q16610

This gene encodes an ˜85 KDa soluble protein that is involved in endochondral bone formation, proliferation of endothelial cells, angiogenesis, and tumour biology. It also interacts with a variety of extracellular and structural proteins, contributing to the maintenance of skin integrity and homeostasis. ECM1 acts as a “biological glue” in a variety of tissues contributing to the organization and scaffolding of collagen.

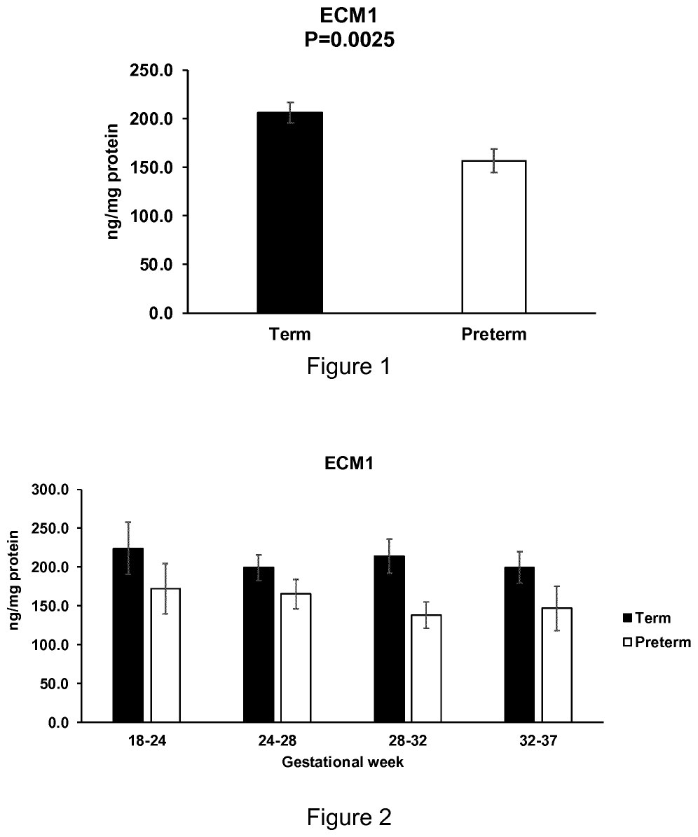

4 alternatively spliced transcript variants encoding distinct isoforms have been described for this gene. Any of these may be used as a biomarker according to the present disclosure. In some cases, variant 1 is detected. As shown in FIG. 1, the overall mean concentration of ECM1 from all samples (collected between 19 and 37 weeks gestation) was lower in samples obtained from individuals that experienced preterm birth than in samples that experienced term birth (p=0.025). This indicates that under-expression of ECM1 in a sample indicates that the individual is at risk of preterm birth.

This is further shown in FIG. 2, in which ECM1 was under-expressed in samples from individuals that experienced preterm birth, at all time points sampled. In an individual suspected at being at risk of preterm birth, a lower level of ECM1 may indicate birth will occur in less than 12 weeks, less than 9 weeks, or less than 4 weeks.

### Gamma-Glutamyl Hydrolase (GGH); UniProtKB-Q92820

GGH hydrolyzes the polyglutamate side chains of pteroylpolyglutamates, which progressively removes gamma-glutamyl residues from pteroylpoly-gamma-glutamate to yield pteroyl-alpha-glutamate (folic acid) and free glutamate. It may play an important role in the bioavailability of dietary pteroylpolyglutamates and in the metabolism of pteroylpolyglutamates and antifolates.

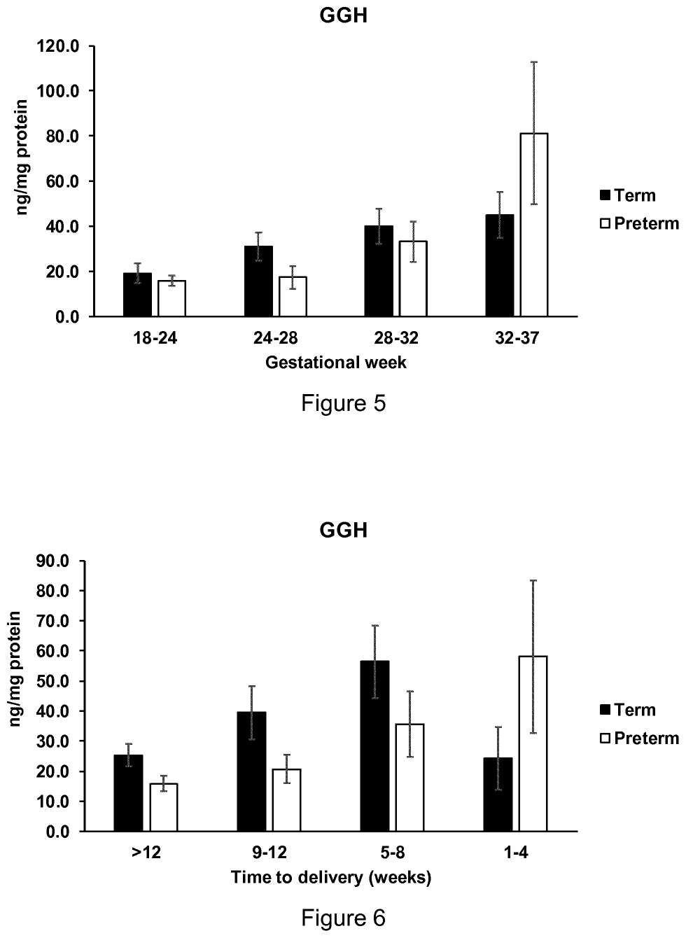

As shown in FIG. 4, the overall mean concentration of GGH from all samples (collected between 19 and 37 weeks gestation) was increased in samples obtained from individuals that experienced preterm birth than in samples that experienced term birth. This indicates that over-expression of GGH in a sample indicates that the individual is at risk of preterm birth. This is particularly apparent from FIGS. 5 and 6 which demonstrate that GGH is particularly over-expressed in samples obtained 1-4 weeks prior to preterm birth, or at 32-37 weeks gestation. This indicates that over-expression of GGH in a sample obtained at 32-37 weeks gestation indicates that the individual is at risk of preterm birth. Conversely, under-expression of GGH in a sample obtained prior to 32 weeks, or at around 26 weeks or less gestation may indicate that the individual is at risk of preterm birth. In an individual suspected of being at risk of preterm birth, an increased level of GGH may indicate that birth will occur in 4 weeks or less.

### Laminin Subunit Gamma-2 (LAMC2); UniProtKB-Q13753,

LAMC2 is a heparin binding protein that binds to the cells via a high affinity receptor. Laminin is thought to mediate the attachment, migration, and organization of cells into tissues during embryonic development by interacting with other extracellular matrix components. Ladsin, a laminin variant containing the laminin gamma-2 chain exerts cell-scattering activity toward a wide variety of cells, including epithelial, endothelial, and fibroblastic cells.

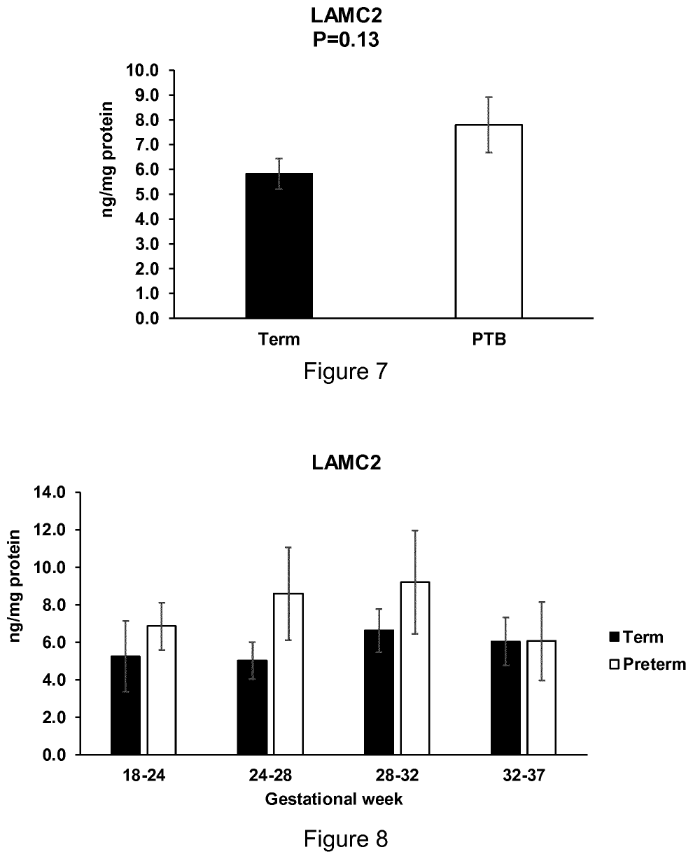

As shown in FIG. 7, LAMC2 was increased in samples obtained from individuals that experienced preterm birth than in samples that experienced term birth. This indicates that over-expression of LAMC2 in a sample indicates that the individual is at risk of preterm birth.

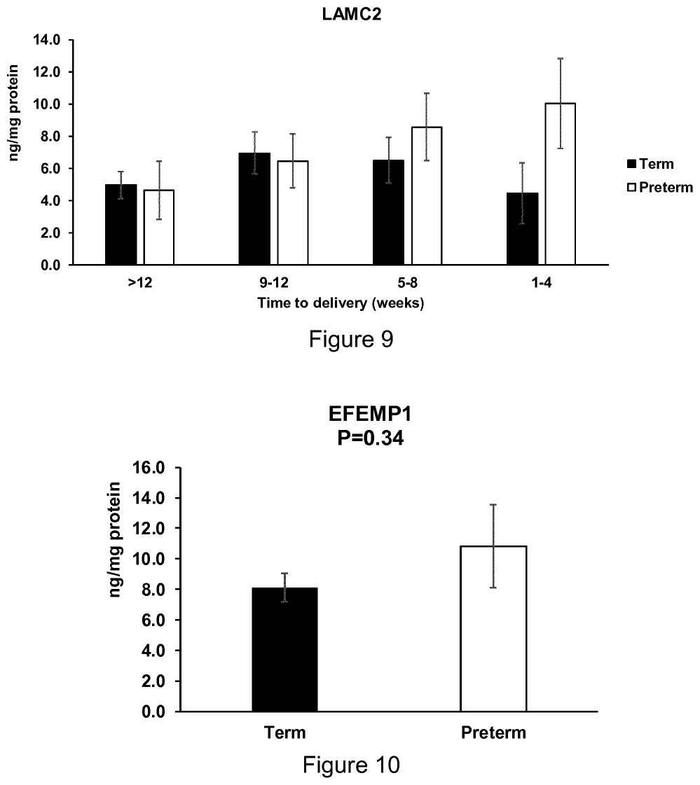

As shown in FIG. 8, LAMC2 was over-expressed in preterm birth samples obtained prior to 32 weeks of gestation or in samples obtained less than 8 weeks before birth in preterm birth samples (FIG. 9).

### EGF Containing Fibulin-Like Extracellular Matrix Protein 1 (EFEMP1); UniProtKB-Q12805

The EFEMP1 protein binds EGFR, the EGF receptor, and induces EGFR autophosphorylation and the activation of downstream signalling pathways. It may play a role in cell adhesion and migration. It may function as a negative regulator of chondrocyte differentiation. In the olfactory epithelium, it may regulate glial cell migration, differentiation, and the ability of glial cells to support neuronal neurite outgrowth.

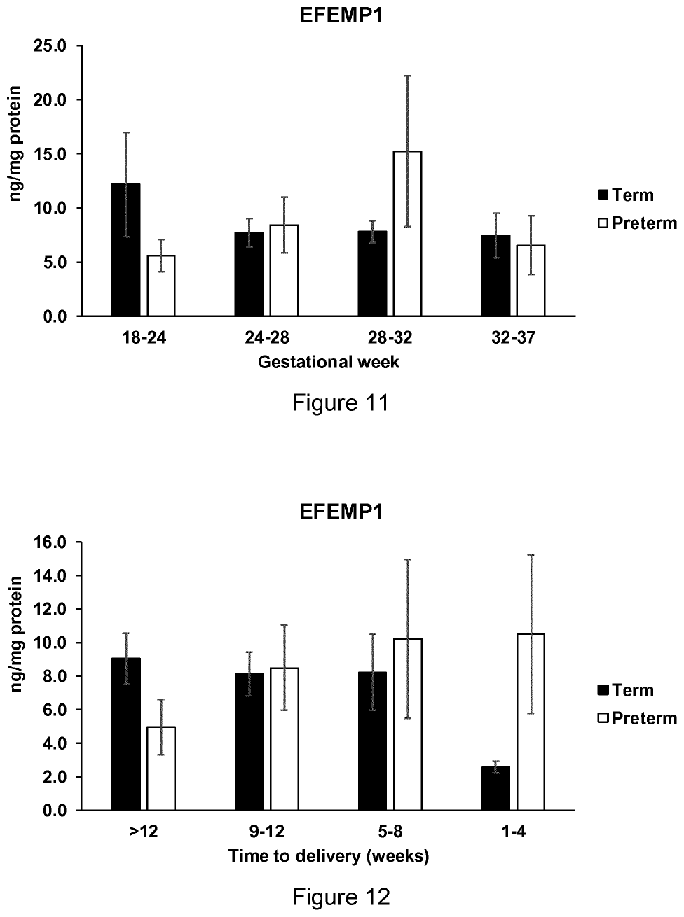

As shown in FIG. 10, EFEMP 1 is increased in samples obtained from individuals that experienced preterm birth than in samples that experienced term birth. This indicates that over-expression of EFEMP1 in a sample indicates that the individual is at risk of preterm birth. This is also apparent from FIGS. 11 and 12, in which EFEMP1 was increased in preterm birth samples up to 8 weeks prior to birth, or from 28 weeks gestation.

Significant upregulation of EFEMP1 in a sample from an individual determined to be at risk of preterm birth may indicate that the birth will occur in the next 1-4 weeks.

Downregulation of EFEMP1 in a sample obtained between 18-24 or 19-26 weeks of gestation may indicate that the individual will experience preterm birth.

### Pleiotrophin (PTN); UniProtKB-P21246

PTN is a secreted growth factor that induces neurite outgrowth, and which is mitogenic for fibroblasts, epithelial, and endothelial cells (PubMed:1768439, PubMed:1733956). It binds to anaplastic lymphoma kinase (ALK), which induces MAPK pathway activation, an important step in the anti-apoptotic signalling of PTN and regulation of cell proliferation (PubMed:11278720). It binds to cell-surface target proteins via their chondroitin sulfate groups (PubMed:26896299). Upon binding, PTN inactivates the phosphatase activity of PTPRZ1.

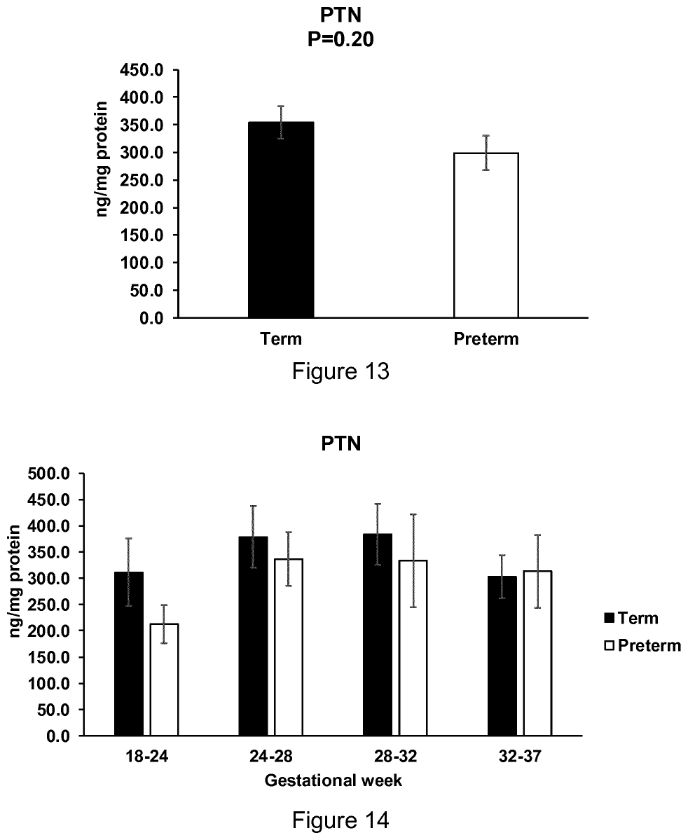

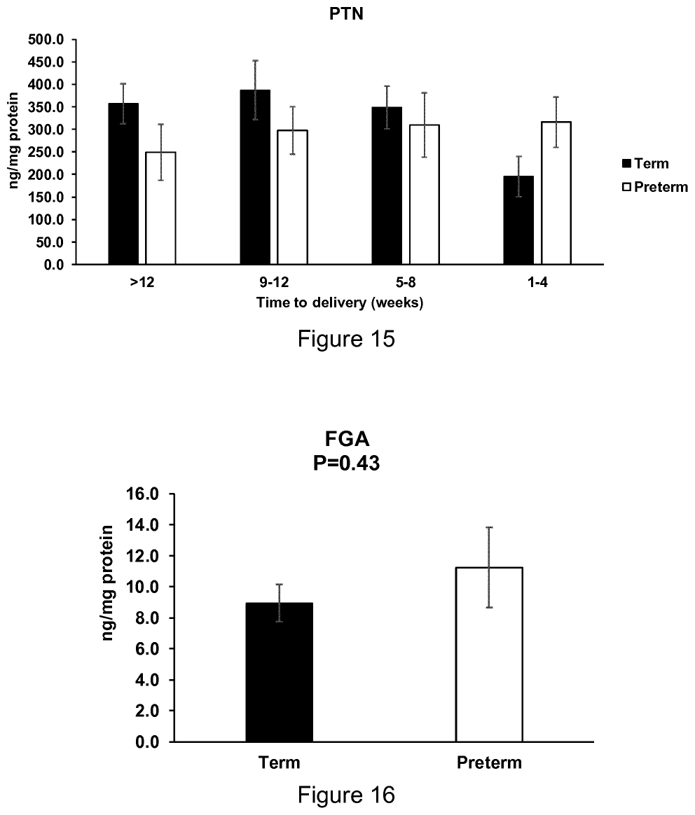

As shown in FIG. 13, PTN is decreased in samples obtained from individuals that experienced preterm birth than in samples that experienced term birth. This suggests that under-expression of PTN in a sample may indicate that the individual is at risk of preterm birth. This is also apparent from FIGS. 14 and 15, with PTN decreased in samples obtained from individuals that experienced preterm birth, sampled prior to 32 weeks of gestational age or more than 4 weeks before delivery.

Overexpression of PTN in a sample may indicate that an individual will experience preterm birth within the next 1-4 weeks.

### Fibrinogen Alpha Chain (FGA); UniProtKB-P02671

FGA is cleaved by the protease thrombin to yield monomers which, together with fibrinogen beta (FGB) and fibrinogen gamma (FGG), polymerize to form an insoluble fibrin matrix. Fibrin has a major function in haemostasis as one of the primary components of blood clots. In addition, it functions during the early stages of wound repair to stabilize the lesion and to guide cell migration during re-epithelialization. FGA was originally thought to be essential for platelet aggregation, based on in vitro studies using anticoagulated blood. However, subsequent studies have shown that it is not absolutely required for thrombus formation in vivo. FGA enhances expression of P-selectin (SELP) in activated platelets via an ITGB3-dependent pathway. Maternal fibrinogen is essential for successful pregnancy. Fibrin deposition is also associated with infection, where it protects against IFNG-mediated haemorrhage. It may also facilitate the immune response via both innate and T-cell mediated pathways.

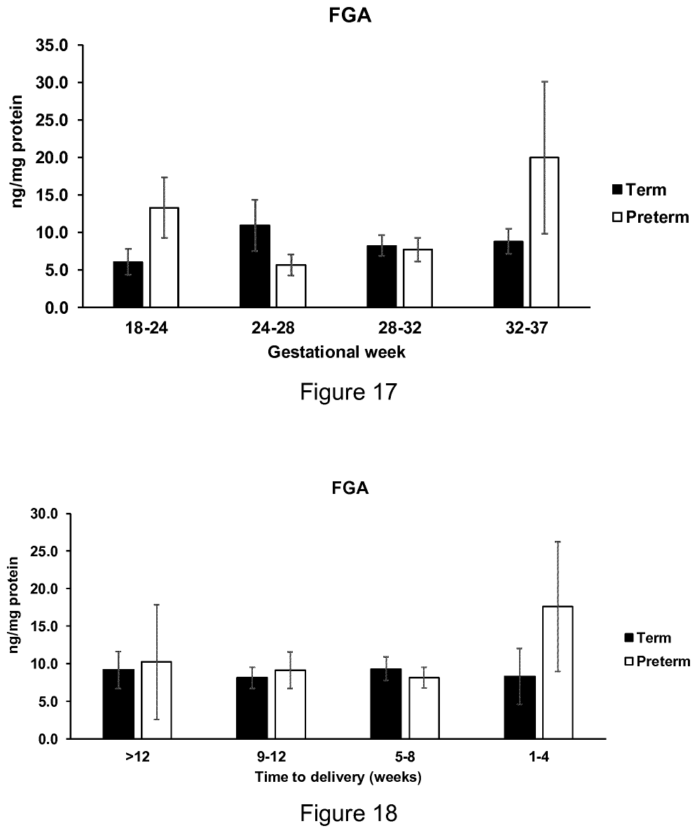

As shown in FIG. 16, FGA is increased in samples obtained from individuals that experienced preterm birth than in samples that experienced term birth. This indicates that over-expression of FGA in a sample indicates that the individual is at risk of preterm birth. This is also apparent from FIGS. 17 and 18, over-expression of FGA in a sample may indicate that the individual is likely to experience preterm birth, particularly when sample was collected before 24 gestational week or after 32 gestational week. Over-expression of FGA may indicate that the individual is likely to experience preterm birth within the next 1-4 weeks.

Underexpression of FGA in a sample collected between 24 and 28 weeks gestation may indicate that the individual is likely to experience preterm birth.

### Pigment Epithelium-Derived Factor (PEDF); UniProtKB-P36955

PEDF is a neurotrophic protein, which induces extensive neuronal differentiation in retinoblastoma cells, as well as a potent inhibitor of angiogenesis. As it does not undergo the S (stressed) to R (relaxed) conformational transition characteristic of active serpins, it exhibits no serine protease inhibitory activity.

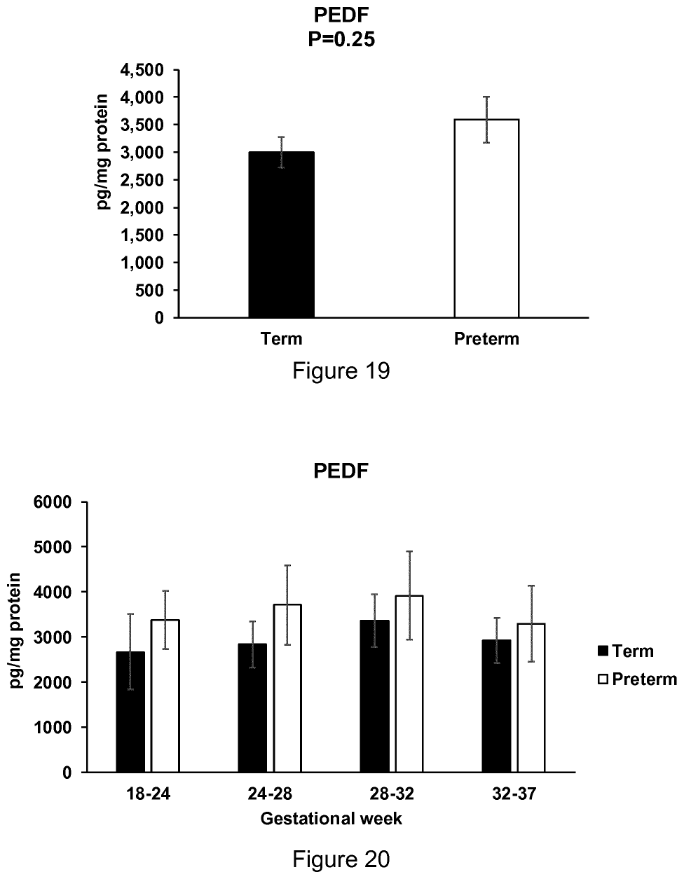

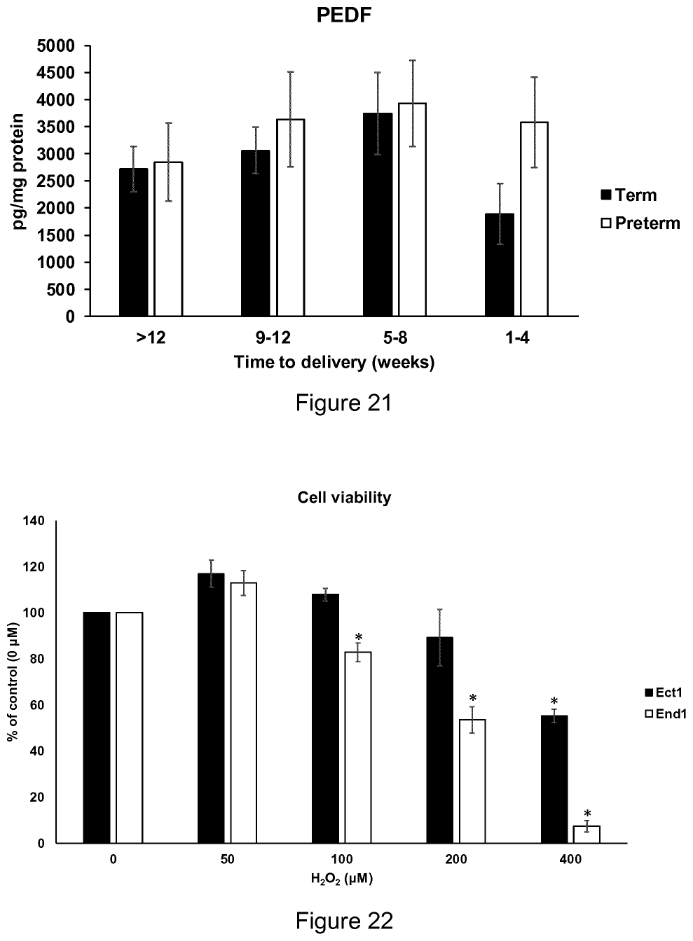

As shown in FIG. 19, PEDF is increased in samples obtained from individuals that experienced preterm birth than in samples that experienced term birth. This indicates that over-expression of PEDF in a sample indicates that the individual is at risk of preterm birth. This is also apparent from FIGS. 20 and 21, over-expression of PEDF in a sample may indicate that the individual is likely to experience preterm birth, irrespective of the sampling time. Over-expression of PEDF may indicate that the individual is likely to experience preterm birth within the next 1-4 weeks.

Certain methods disclosed herein involve detecting a biomarker value or biomarker level. These terms refer to a measurement that is made using any appropriate analytical method for detecting the biomarker in a biological sample and that indicates the presence, absence, absolute amount or concentration, relative amount or concentration, titer, level, expression level, ratio or other measurement corresponding to the biomarker in the sample. The exact nature of the value or level depends on the specific design and components of the particular analytical method employed to detect the biomarker.

Biomarkers that indicate that an individual is at risk of preterm birth may be over-expressed or under-expressed, as compared to a reference value or level or the biomarker that indicates or is a sign of term birth. “up-regulation”, “over-expression”, increased and related terms are used to refer to a value or level in a sample that is greater than a value or level (or range of values or levels) of the biomarker that is typically detected in similar samples from individuals that are known to have experienced term birth.

“down-regulation”, “under-expression”, “reduced” and related terms are used to refer to a value or level in a sample that is less than a value or level (or range of values or levels) of the biomarker that is typically detected in similar samples from individuals that are known to have experienced term birth.

A biomarker that is over-expressed or under-expressed may also be referred to as being “differentially expressed” or as having a “differential” level or value as compared to the expression level or value observed in individuals known to have experienced term birth. Differential expression can also be referred to as a variation from a “normal” expression level of the biomarker.

The term “differential gene expression” and “differential expression” are used interchangeably to refer to a gene (or its corresponding protein expression product) whose expression is activated to a higher or lower level in a subject at risk of preterm birth, relative to its expression in an individual known to have experienced term birth. The terms also include genes (or the corresponding protein expression products) whose expression is activated to a higher or lower level at different stages of the same disease. It is also understood that a differentially expressed gene may be either activated or inhibited at the nucleic acid level or protein level, or may be subject to alternative splicing to result in a different polypeptide product. Such differences may be evidenced by a variety of changes including mRNA levels, surface expression, secretion or other partitioning of a polypeptide. Differential gene expression may include a comparison of expression between two or more genes or their gene products; or a comparison of the ratios of the expression between two or more genes or their gene products; or even a comparison of two differently processed products of the same gene, which differ between individuals at risk of preterm birth or individuals that experience term birth. Differential expression includes both quantitative, as well as qualitative, differences in the temporal or cellular expression pattern in a gene or its expression products in individuals who experience preterm and term birth.

### Method Outcomes

Methods disclosed herein are useful for identifying individuals at risk of preterm birth, or for determining whether an individual is, or is not, at risk of preterm birth. Methods may also be used to predict the risk of preterm birth in an individual.

In some cases, the method may involve a step of recording the level of the biomarkers. In some cases, the methods may involve a step of transmitting the level of the biomarkers disclosed herein to a physician involved in the care of the individual being tested. In some cases, the method may also involve transmitting a reference level of the biomarker, for comparison with the level of biomarker in the individual. In some cases, the level of risk of preterm birth determined in the individual is transmitted to the physician. For example, a level of risk may be allocated a percentage (where 100% indicates that the individual will certainly experience preterm birth, and 0% indicates that the individual will certainly experience term birth). Thus, some methods disclosed herein involve allocating a percentage value to the level of risk that the individual is determined to have. The method may involve the step of transmitting that percentage to a physician involved in the care of that individual.

The methods disclosed herein may be used to select an individual for treatment or other management. Certain methods disclosed herein involve the administration of a treatment to an individual identified as at risk of preterm birth.

Treatments useful in the methods disclosed herein include the administration of a progesterone, synthetic progesterone or progesterone analogue, an one or more agents selected from a progesterone or an analogue thereof, a tocolytic, a corticosteroid, an antibiotic, an NSAID or an Omega 3 fatty acid or derivative thereof. The progesterone may be a synthetic progesterone, such as 17-a-hydroxyprogesterone caproate. The tocolytic may be magnesium sulfate, indomethacin or Nifedipine.

The antibiotic may be erythromycin or penicillin. The NSAID may be indomethacin. The Omega 3 fatty acid derivative may be docosahexaenoic acid (DHA).

The individual may be selected for treatment with progesterone. Progesterone has been shown to reduce preterm birth rates in numerous clinical studies profiling women with short cervical length or prior history of preterm birth as a high-risk population. Treatment with progesterone may comprise the administration of natural progesterone, or synthetic progestin such as 17-α-hydroxyprogesterone caproate. The progesterone may be P4 micronized (natural) progesterone. 17-α-hydroxyprogesterone caproate is also known by the brand names Delalutin™, Proluton™ Proluton Depot™ and Makena™. Natural micronized progesterone, a natural progesterone, is similar to that produced in the corpus luteum and placenta. Micronized progesterone can be utilised as oral capsule, vaginal gel or vaginal suppository. Synthetic Progestins include medroxyprogesterone acetate (MPA, also known as depot medroxyprogesterone acetate (DMPA)) and norethindrone acetate (NETA). They are typically given by injection. Syntethic progestins are also known by the brand names (MPA) Provera™ Depo-Provera™, Depo-SubQ Provera 104™ Curretab™ Cycrin™, Farlutal™, Gestapuran™, Perlutex™, Veramix™ and (NETA) Primolut-Nor™, Aygestin™ Gestakadin™ Milligynon™, Monogest™, Norlutate™, Primolut N™, SH420™, Sovel™, Styptin™. The micronized progesterone may be self-administered by the patient. Natural micronized progesterone is also known by the brand names Prometrium™ Utrogestan™, Endometrin™ and Crinone™. Administration may be orally, vaginally, or intramuscularly. Progesterone, progestin or 17-α-hydroxyprogesterone caproate for use in such methods, or the use of progesterone, progestin or 17-α-hydroxyprogesterone caproate for use in the manufacture of a medicament for use in such a method are also disclosed.

Individuals identified at risk of preterm birth may be treated with cervical cerclage. Cervical cerclage may also be referred to as a cervical stitch. Cervical cerclage is used to treat cervical incompetence or insufficiency, where the cervix starts to shorten and open too early during a pregnancy. Cervical cerclage may involve the insertion of a strong suture into and around the cervix.

Any known form of cervical cerclage may be used. For example, the cervical cerclage may be a McDonald cerclage, a Shirodkar cerclage or an abdominal cerclage. Cervical cerclage may be particularly useful where the individual is determined to have cervical incompetence. Cervical incompetence may be determined by transvaginal ultrasound scan.

Alternatively, the treatment may comprise a cervical pessary. In some cases, the treatment may be an Arabin Pessary.

In some cases, the individual may be selected to receive tocolytics or steroids, such as corticosteroids. Tocoloytics may be used to arrest uterine contraction during preterm labor. Steroids may aid in fetal lung development. The method may involve a step of administering the tocolytic or steroid to the individual. Tocolytics and steroids have been used for women presenting with contractions. Examples of tocolytic agents suitable in the invention are magnesium sulfate, indomethacin and nifedipine.

In some cases, the methods are used to select an individual for further, regular or intensive monitoring. For example, the methods may be used to determine that a further sample should be obtained from that individual, and the biomarker presence or absence and/or level should be determined in the future. The further sample may be obtained 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 weeks after the first sample. The further sample may be obtained at gestational week 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 27. The method may involve further sampling every 1, 2, 3, 4, or 5 weeks during the pregnancy.

In some cases, the individual may be treated with antibiotics. Antibiotics may be particularly used in individuals with preterm premature rupture of the membranes (PPROM). Suitable antibiotics include erythromycin and penicillin.

In some cases, the treatment may be administration of an NSAID. NSAIDs may inhibit prostaglandin to reduce uterine contractions. The NSAID may be indomethacin. In some cases, the treatment may be Omega 3 Fatty Acid or a derivative of Omega 3 Fatty acid. For example, the treatment may be DHA (docosahexaenoic acid).

Monitoring may comprise monitoring for fetal distress, such as monitoring fetal heartbeat, monitoring fetal movement or meconium monitoring.

### Measurement of Biomarkers

Biomarkers disclosed herein are preferably protein biomarkers. Any method of detecting and/or quantifying a protein known in the art may be used.

Methods according to the present invention may be performed, or products may be present, in vitro, ex vivo, or in vivo. The term “in vitro” is intended to encompass experiments with materials, biological substances, cells and/or tissues in laboratory conditions or in culture whereas the term “in vivo” is intended to encompass experiments and procedures with intact multi-cellular organisms. “Ex vivo” refers to something present or taking place outside an organism, e.g. outside the human or animal body, which may be on tissue (e.g. whole organs) or cells taken from the organism.

The methods disclosed herein relate to the determination of protein expression. Protein expression can be measured by quantifying the amount of protein in a cell, tissue or sample, or by observing the localisation of the protein within cells and tissues.

In some cases, immunoassays are used to detect the biomarker target in a sample from the subject. Immunoassays use antibodies or other entities with specific affinity for the target molecule in conjunction with a detectable molecule. In some cases, the antibody is conjugated to the detectable molecule. The detectable molecule may be referred to as a label. The detectable molecule produces a detectable signal when the antibody is bound to the target molecule. The detectable signal may be a quantifiable signal. In some cases, an aptamer is used instead of, or together with, the antibody. Immunoassays include ELISA, immunoblotting, flow cytometry and immunohistochemistry. In certain aspects described herein, the assay is an immunohistochemistry assay. Such assays commonly use antibodies, although other target specific molecules such as aptamers or other ligands may be used. Antibody arrays or protein chips may also be used.

The method may be approved for use by a regulatory agency. The method may be an FDA approved method.

### Antibodies

Antibodies which will bind to the biomarkers of the invention are already known. In view of today's techniques in relation to monoclonal antibody technology, antibodies can be prepared to most antigens.

The antigen-binding portion may be a part of an antibody (for example a Fab fragment) or a synthetic antibody fragment (for example a single chain Fv fragment [ScFv]). Suitable monoclonal antibodies to selected antigens may be prepared by known techniques, for example those disclosed in “Monoclonal Antibodies: A manual of techniques”, H Zola (CRC Press, 1988) and in “Monoclonal Hybridoma Antibodies: Techniques and Applications”, J G R Hurrell (CRC Press, 1982). Chimeric antibodies are discussed by Neuberger et al (1988, 8th International Biotechnology Symposium Part 2, 792-799).

Monoclonal antibodies (mAbs) are useful in the methods of the invention and are a homogenous population of antibodies specifically targeting a single epitope on an antigen. Suitable monoclonal antibodies can be prepared using methods well known in the art (e.g. see Köhler, G.; Milstein, C. (1975). “Continuous cultures of fused cells secreting antibody of predefined specificity”. Nature 256 (5517): 495; Siegel D L (2002). “Recombinant monoclonal antibody technology”. Schmitz U, Versmold A, Kaufmann P, Frank H G (2000); “Phage display: a molecular tool for the generation of antibodies—a review”. Placenta. 21 Suppl A: S106-12. Helen E. Chadd and Steven M. Chamow; “Therapeutic antibody expression technology,” Current Opinion in Biotechnology 12, no. 2 (Apr. 1, 2001): 188-194; McCafferty, J.; Griffiths, A.; Winter, G.; Chiswell, D. (1990). “Phage antibodies: filamentous phage displaying antibody variable domains”. Nature 348 (6301): 552-554; “Monoclonal Antibodies: A manual of techniques”, H Zola (CRC Press, 1988) and in “Monoclonal Hybridoma Antibodies: Techniques and Applications”, J G R Hurrell (CRC Press, 1982). Chimaeric antibodies are discussed by Neuberger et al (1988, 8th International Biotechnology Symposium Part 2, 792-799)).

Polyclonal antibodies are useful in the methods of the invention. Monospecific polyclonal antibodies are preferred. Suitable polyclonal antibodies can be prepared using methods well known in the art.

Fragments of antibodies, such as Fab and Fab2 fragments may also be used as may genetically engineered antibodies and antibody fragments. The variable heavy (VH) and variable light (VL) domains of the antibody are involved in antigen recognition, a fact first recognised by early protease digestion experiments. Further confirmation was found by “humanisation” of rodent antibodies. Variable domains of rodent origin may be fused to constant domains of human origin such that the resultant antibody retains the antigenic specificity of the rodent parented antibody (Morrison et al (1984) Proc. Natl. Acad. Sd. USA 81, 6851-6855).

That antigenic specificity is conferred by variable domains and is independent of the constant domains known from experiments involving the bacterial expression of antibody fragments, all containing one or more variable domains. These molecules include Fab-like molecules (Better et al (1988) Science 240, 1041); Fv molecules (Skerra et al (1988) Science 240, 1038); single-chain Fv (ScFv) molecules where the VH and VL partner domains are linked via a flexible oligopeptide (Bird et al (1988) Science 242, 423; Huston et al (1988) Proc. Natl. Acad. Sd. USA 85, 5879) and single domain antibodies (dAbs) comprising isolated V domains (Ward et al (1989) Nature 341, 544). A general review of the techniques involved in the synthesis of antibody fragments which retain their specific binding sites is to be found in Winter & Milstein (1991) Nature 349, 293-299.

By “ScFv molecules” we mean molecules wherein the VH and VL partner domains are covalently linked, e.g. directly, by a peptide or by a flexible oligopeptide.

Fab, Fv, ScFv and dAb antibody fragments can all be expressed in and secreted from E. coli, thus allowing the facile production of large amounts of the said fragments.

Whole antibodies, and F(ab′)2 fragments are “bivalent”. By “bivalent” we mean that the said antibodies and F(ab′)2 fragments have two antigen combining sites. In contrast, Fab, Fv, ScFv and dAb fragments are monovalent, having only one antigen combining site. Synthetic antibodies which bind to the biomarker may also be made using phage display technology as is well known in the art (e.g. see “Phage display: a molecular tool for the generation of antibodies—a review”. Placenta. 21 Suppl A: S106-12. Helen E. Chadd and Steven M. Chamow; “Phage antibodies: filamentous phage displaying antibody variable domains”. Nature 348 (6301): 552-554).

In some preferred embodiments the antibody is detectably labelled or, at least, capable of detection. For example, the antibody may be labelled with a radioactive atom or a coloured molecule (chromophore) or a fluorescent molecule or a molecule which can be readily detected in any other way. Suitable detectable molecules include fluorescent proteins, luciferase, enzyme substrates, and radiolabels. The antibody may be directly labelled with a detectable label or it may be indirectly labelled. For example, the antibody may be unlabelled and can be detected by another antibody which is itself labelled. Alternatively, the second antibody may have bound to it biotin and binding of labelled streptavidin to the biotin is used to indirectly label the first antibody.

An aspect disclosed herein is a complex of an antibody and a biomarker selected from the group consisting of ECM1, FGA, EFEMP1, GGH, PEDF, PTN and LAMC2. The complex may further comprise a second, different antibody. The complex may further comprise a detectable moiety. The complex may be present in a sample of cervicovaginal fluid. The complex may be isolated.

### Detection and Labelling

Methods disclosed herein involve the detection and/or quantification of a biomarker.

In the methods disclosed herein, the biomarker (the “target”) may be directly detected. That is to say that the target is detected by an anti-target antibody.

Alternatively, detection of the target may be indirect. That is to say that the target may be detected by the anti-target antibody, and the anti-target antibody is subsequently detected by a secondary detectable antibody. The secondary antibody is preferably labelled. Suitable secondary antibodies may be raised against the antibody isotype of the animal species in which the primary antibody has been raised. For example, the secondary antibody may be an anti-mouse antibody, capable of binding to mouse antibodies. Methods using a secondary antibody may be more sensitive than direct detection methods, due to signal amplification from multiple secondary antibodies binding to each primary antibody.

Suitable labels include enzymes such as horseradish peroxidase, alkaline phosphatase, glucose oxidase and luciferase, and colorimetric agents, including quantum dots, fluorophores and chromophores. Suitable fluorophores include FITC, TRITC, Cy5, Texas Red, Alexa Fluor and others. The label may be a radiolabel.

A variety of detectable enzymatic substrates are available for use with enzymatically labelled antibodies. These include chromogenic detection systems, such as Horseradish peroxidase (HRP), pNNP, BCIP/NBT (5-bromo-4-chloro-3′-indolyphosphate/nitro-blue tetrazolium), TMB (tetramethybenzidine), DAB (3,3′-diaminobenzidine), OPD (ortho-phenylenediaine dihydrochloride) and ABTS (2,2′-azinobis[-ethylbenzothiazoline-6-sulfonic acid]), and chemiluminscent substrates such as an ECL (enhanced chemiluminscent) label or Acridinium ester (AE).

Methods may involve the use of an antibody or antibody-derived binding agent, such as a scFv or Fab fragment. Alternatively, or in combination with the antibody, the method may involve the use of an aptamer.

### ELISA

In some cases, the target may be detected by ELISA (enzyme-linked immunosorbent assay). Target molecules (such as the biomarker proteins disclosed herein) from a sample are attached to a surface and detected using a specific antibody. The target may be attached to the surface non-specifically (via adsorption to the surface) or specifically (using a specific capture agent such as an antibody). ELISA may be used to quantify target in a sample. The surface may be a solid support, such as a multiwall plate, microbead, or dipstick.

Commercially available ELISA assays may be used. The ELISA may be an indirect ELISA, Sandwich ELISA or competitive ELISA.

ELISA involves the use of first, capture, antibody to bind the target molecule. A second, detection, antibody to the target molecule is then added. Binding of the second antibody indicates the presence and/or level of the target.

The first antibody may be bound to a solid support. The first and second antibodies are not identical. Usually, the first and second antibodies bind to different epitopes on the target molecule. In some cases, the second antibody binds to a complex of the first antibody and the target, but not to either the first antibody or the target when not in complex. The second antibody may be labelled.

### Immunoblottinq

In some aspects, the target is detected by immunoblotting, or western blotting. In such methods, proteins in a sample are separated based on their electric charge or size. They may be separated by an electrophoresis-based method. The separated proteins are transferred to a membrane, where they are stained with an antibody that is specific to the target. The antibody is then detected, either directly by virtue of the antibody being conjugated to a detectable label, or indirectly, by adding a labelled secondary antibody.

### Mass Spectrometry

In some aspects, the methods disclosed herein involve the detection and/or quantification of protein using mass spectrometry. Mass spectrometry may use peptides with sequences unique to the target protein as surrogates for the target. Measurements are made with respect to the mass and intensity of the peak due to the protein, protein fragment or partial peptide of interest. Prior to the measurements a fixed amount of substance serving as the internal standard is added to the original biological material and the intensity of its peak is also measured. The concentration of the target in the original biological material can be calculated from the ratio of peak intensity of the target to the peak intensity of the internal standard. Various mass-spectrometry methods are known and may be used for detecting and/or quantifying biomarkers as disclosed herein, including MALDI-TOF (time of flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, or tandem mass spectrometry.

### In Vitro Diagnostics and Kits

An aspect of the present disclosure includes in vitro diagnostic methods, and in vitro diagnostic kits for performing such methods. A kit as described herein may include one or more antibodies, such as an anti-biomarker antibody or fragment thereof. The kit may be suitable for selecting a subject at risk of preterm birth.

The kit may be suitable for a point-of-care in vitro diagnostic test. It may be kit for laboratory-based testing. The kit may include instructions for use, such as an instruction booklet or leaflet. The instructions may include a protocol for performing any one or more of the methods described herein. The instructions may include a protocol for performing an immunochromatographic assay. They may describe methods and suggestions for adapting the test for different types of sample. They may provide methods and suggestions for optimising the results obtained from the test, such as minimising the signal to noise ratio.

The kit may be suitable for performing an immunochromatographic assay. In some cases, the in vitro diagnostic test involves a lateral flow device, or “dipstick” test. In some cases, the kit includes a multiwell plate or other solid support that is pre-coated with a capture agent, such as an anti-biomarker antibody.

The kit may additionally include standards or controls. The kit may additionally include buffers, diluents or other reagents, such as stop buffer, sample preparation buffer, colour development reagents, streptavidin conjugates, substrates or wash buffer.

The kit may be adapted for use with dry samples, wet samples, frozen samples, fixed samples, urine samples, saliva samples, tissue samples, blood samples, or any other type of sample, including any of the sample types disclosed herein.

The kit may comprise a device for obtaining or processing a vaginal fluid sample. The kit may comprise vaginal fluid extraction buffer, for example a buffer containing approximately 50 mM HEPES, 150 mM NaCl, 0.1% SDS, 1 mM EDTA, 1 mM Pefabloc SC 4-(2-aminoethyl_benzene sulfonyl fluoride (AEBSF). The kit may comprise a sample collection device, such as a swab, cervicovaginal wick, diaphragm-like device, cervical aspirator, or cytobrush. The kit may comprise a container suitable for storing a vaginal fluid sample.

Swabs suitable for use in the kits include foam swabs, Dacron swabs, rayon swabs, flocked swabs and cotton swabs. Suitable foam swabs include MW942 (Sigma-Swab Duo), Polyurethane foam swab (Catch-All; Epicenter) and CultureSwab EZ polyurethane foam swabs (BD). Suitable Dacron swabs include Deltalab Eurotubo 300263 (Fisher Scientific, UK), Sterile G-in, Dacron-tipped plastic applicators (Solon, Skowhegan, Me.), Dacron swab (Cardinal Health, McGraw Park, Ill.) and Dacron swabs (Puritan Medical, Guilford, Me., USA). Suitable rayon swabs include BBL CultureSwab (Becton Dickinson, Oxford, UK) and MW167 (Duo-Transtube®). Suitable flocked swabs (nylon) include Seacliff Packaging, BD, COPAN. Suitable cotton swabs include Sterile dry swabs (Eurotubo, Rubi, Spain), Cotton-tipped swabs (Falcon™ Screw Cap Single SWUBE™ applicator, Becton Dickinson and Co., Sparks, Md.), Falcon™ Screw Cap Single SWUBE™ applicator (BD).

Wicks suitable for use in the kit include tampons, strips or sponges, including opthalmic PVA sponge (Eyetec™ Network Medical Ltd.), Tear-Flo™ Strips (Wilson Ophthalmic), Weck-Cel® sponges (Xomed Surgical Products, Jacksonville, Fla.), Sno-strips (Akorn Inc., Abita Springs La.) and Polywicks (Polyfiltronics, Rockland, Mass., USA).

Diaphragm like devices suitable for use in the kit include Instead SoftCup (Ultrafem), Sterile gauze, or a Menstrual cup (SoftCup, EuroFemPro, Netherlands, or the SoftCup, Instead Inc., San Diego, Calif.).

Suitable cervical aspirators include Vaginal Specimen Aspirators (CarTika), or long tuberculin syringes.

Certain kits disclosed herein comprise an antibody that binds to a biomarker of preterm birth and a device or buffer for obtaining or processing a vaginal fluid sample.

The antibody that binds to a biomarker of preterm birth may be an anti-ECM1, anti-GGH, anti-LAMC2, anti-EFEMP1, anti-PTN, anti-FGA or anti-PEDF antibody.

Also disclosed herein is a composition comprising vaginal fluid and an anti-ECM1, anti-GGH, anti-LAMC2, anti-EFEMP1, anti-PTN, anti-FGA or anti-PEDF antibody.

### Sampling Methods

Methods and agents described herein involve the analysis of certain biomarkers in cervicovaginal fluid. Several methods of sampling cervicovaginal fluid are known, and may be used in the methods.

The methods may involve sampling by cervicovaginal lavage. This involves obtaining cervicovaginal washings by rinsing the cervicovagina with washing buffer and collecting the fluid after the rinsing.

In some methods, cervicovaginal swabs are taken. Suitable swabs are known in the art. Preferred swabs for use in the methods and kits disclosed herein include foam swabs, Dacron swabs, rayon swabs, flocked swabs and cotton swabs. Suitable foam swabs include MW942 (Sigma-Swab Duo), Polyurethane foam swab (Catch-All; Epicenter) and CultureSwab EZ polyurethane foam swabs (BD). Suitable Dacron swabs include Deltalab Eurotubo 300263 (Fisher Scientific, UK), Sterile G-in, Dacron-tipped plastic applicators (Solon, Skowhegan, Me.), Dacron swab (Cardinal Health, McGraw Park, Ill.) and Dacron swabs (Puritan Medical, Guilford, Me., USA). Suitable rayon swabs include BBL CultureSwab (Becton Dickinson, Oxford, UK) and MW167 (Duo-Transtube®). Suitable flocked swabs (nylon) include Seacliff Packaging, BD, COPAN. Suitable cotton swabs include Sterile dry swabs (Eurotubo, Rubi, Spain), Cotton-tipped swabs (Falcon™ Screw Cap Single SWUBE™ applicator, Becton Dickinson and Co., Sparks, Md.), Falcon™ Screw Cap Single SWUBE™ applicator (BD).

In other methods, cervicovaginal fluid is sampled with a wick. Wicks suitable for use in the methods disclosed herein include tampons, strips or sponges, including opthalmic PVA sponge (Eyetec™ Network Medical Ltd.), Tear-Flo™ Strips (Wilson Ophthalmic), Weck-Cel® sponges (Xomed Surgical Products, Jacksonville, Fla.), Sno-strips (Akorn Inc., Abita Springs La.) and Polywicks (Polyfiltronics, Rockland, Mass., USA).

In other methods, diaphragm like devices are used to sample cervicovaginal fluid. Suitable diaphragm like devices are placed over the cervix to collect the cervicovaginal fluid and include Instead SoftCup (Ultrafem), Sterile gauze, or a Menstrual cup (SoftCup, EuroFemPro, Netherlands, or the SoftCup, Instead Inc., San Diego, Calif.).

The method may involve the use of a cervical aspirators such as a Vaginal Specimen Aspirators (CarTika), or long tuberculin syringe.

In some methods, cervicovaginal fluid is sampled with a cytobrush.

Certain kits disclosed herein comprise an antibody that binds to a biomarker of preterm birth and a device or buffer for obtaining or processing a vaginal fluid sample.

### Controls

In some methods disclosed herein the level of the biomarker is compared to the level of a control or a reference value or level.

In some cases, the control may be a reference sample or reference dataset. The reference may be derived from one or more samples that have been previously obtained from a subject known to have undergone preterm birth. Alternatively, the reference may be derived from one or more samples that have been previously obtained from a subject known to have undergone term birth. The reference may be a dataset obtained from analyzing a reference sample.

Controls may be positive controls in which the target molecule is known to be present, or expressed at high level, or negative controls in which the target molecule is known to be absent or expressed at low level.

The control may be a sample or level from a patient known to have experienced a preterm or term birth. The control value may be obtained by performing analysis of the biomarker in parallel with a sample from the individual to be tested. Alternatively, the control value may be obtained from a database or other previously obtained value.

### Samples

Methods disclosed herein relate to the detection of biomarkers in a sample obtained from an individual or patient. The method may be performed in vitro. Preferably, the method involves a sample that has been obtained from an individual. Thus, the method may, but preferably does not, involve a step of obtaining a sample from an individual.

Preferably, the sample is a sample of vaginal fluid, such as cervicovaginal (cervico-vaginal; cervical-vaginal) fluid (CVF) or cervical fluid. Alternatively, the sample may be blood sample, such as whole blood, plasma or serum sample, a lymph sample, a urine sample or an amniotic fluid sample. The sample may be a protein sample derived from a vaginal fluid or cervicovaginal fluid sample, or a protein sample derived from a blood sample, such as whole blood, plasma or serum sample, a lymph sample, a urine sample or an amniotic fluid sample.

The sample may have been pre-treated. For example, the sample may have been contacted with one or more preservative agents or buffers. The sample may have been frozen, lyophilized, or dried.

Although the individual or patient may be mammalian, such as a cat, dog, horse, or ape, the individual is preferably a human. The terms “patient”, “individual” and “subject” are used interchangeably herein.

The individual may be a female individual. The individual may be pregnant. The individual may be symptomatic or asymptomatic. Preferably, the individual is asymptomatic.

Symptomatic individuals are individuals who present with one or more symptoms of preterm birth, such as contractions, particularly regular contractions, back ache, including back ache in the lower back, cramping in the lower abdomen or menstrual-like cramps, fluid leaking from the vagina, flu-like symptoms, nausea, vomiting, increased pressure in the pelvis or vagina, increased vaginal discharge and/or vaginal bleeding.

Asymptomatic individuals may not present with any symptoms of preterm birth, or with symptoms that may or may not be indicative of preterm birth, such as backache, including backache in the lower back, cramping in the lower abdomen or menstrual-like cramps, fluid leaking from the vagina, flu-like symptoms, nausea, vomiting, increased pressure in the pelvis or vagina, increased vaginal discharge and/or vaginal bleeding. Commonly, asymptomatic individuals do not present with any symptom of preterm birth.

In some cases, the individual may be suspected of being at high risk of preterm birth prior to obtaining the sample. For example, the sample may be obtained and/or the presence or level of the biomarker may be determined because the individual is suspected to be of high risk of preterm birth. An individual may be suspected to have a high risk of preterm birth based on their prior medical history of premature births or miscarriages. Alternatively, or additionally, the individual may be suspected to have high risk of preterm birth based on the results of a Fetal Fibronectin (fFN) test, or based on symptoms such as contractions, vaginal bleeding, fluid leaking from the vagina, increased vaginal discharge, backache or cramping in lower abdomen. Alternatively, or additionally, the individual may be considered to be of high risk of preterm birth due to the presence of one or more risk factors such as diabetes, high blood pressure, being pregnant with more than one baby, BMI (too high or too low), a number of vaginal infections, tobacco smoking, psychological stress, ethnic background, and socioeconomic status or income.

Samples may be obtained from an individual weeks or months prior to birth, or prior to the expected date of term birth. For example, samples may be obtained 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 weeks prior to birth. In some cases, samples are taken 1-4, 5-8. 9-12 or more than 12 weeks prior to birth.

Samples may be obtained at a time point which, based on a 37-week expected term, is predicted to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 weeks prior to normal birth. In some cases, samples are taken 1-4, 5-8. 9-12 or more than 12 weeks prior to the expected normal birth date.

Alternatively, samples may be taken at around 1 month, around 2 months, around 3 months, around 4 months, around 5 months, around 6 months, around 7 months, around 8 months, or around 9 months prior to the expected normal birth date.

Looked at another way, samples may be taken at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or 37 weeks of gestation.

Samples may be taken 10 weeks to 13 weeks plus 6 days, 14 weeks to 21 weeks plus 6 days, 22 weeks to 25 weeks plus 6 days, 26 weeks to 29 weeks plus 6 days, 30 weeks to 33 weeks plus 6 days, or more than 34 weeks gestational age. The first sample may be taken around 12-14 weeks. The second sample may be taken between 16-24 weeks.

Samples may be taken in the first, second or third trimester. The first trimester lasts from zero to 13 weeks plus 6 days. The second trimester lasts from 14 weeks to 27 weeks plus 6 days. The third trimester lasts from 28 weeks until birth.

The skilled person will appreciate that it may be difficult to precisely determine the number of weeks of gestation. Methods for estimating the number of weeks of gestation are known in the art, and any of these may be used in the methods disclosed herein. For example, weeks of gestation are commonly estimated based on the date of the last menstrual period (LMP). The weeks of gestation may be determined based on the date on which the last menstrual period began. Alternatively, weeks of gestation may be based on the date of ovulation, if known. Commonly, the date of ovulation is two weeks after the date on which the last menstrual period began. Length of gestation may be determined based on a dating scan. A dating scan is commonly performed between 10 and 13 weeks plus 6 days, based on the date of the first day of the last menstrual period.

Different biomarkers may be more appropriate at different sample times. For example, a biomarker may be useful for determining whether an individual is at risk of preterm birth in a sample obtained from that individual at an early stage, whereas a different biomarker may be useful for determining that an individual is at risk in a sample obtained from that individual at a later stage.

In some cases, samples may be obtained from an individual at multiple time points. For example, a first sample may be obtained in the first trimester, and a second sample may be obtained in the second trimester. Multiple samples may be obtained in order to identify trends or changes in biomarker expression. In some cases, a sample may be obtained early in the pregnancy, such as in the first trimester, so as to establish a control or baseline level of biomarker for that individual.

### Proteins and Polypeptides

Whilst the methods of the present invention may involve the detection of full-length protein sequences, this is not always necessary. As an alternative, homologues, mutants, derivatives, isoforms, splice-variants or fragments of the full-length polypeptide may be detected.

Derivatives include variants of a given full-length protein sequence and include naturally occurring allelic variants and synthetic variants which have substantial amino acid sequence identity to the full-length protein.

Protein fragments may be up to 10, 20, 30, 40, 50, 60, 70, 80, 90, 100 or 150 amino acid residues long. Minimum fragment length may be 3, 4, 5, 6, 7, 8, 9, 10, 11, 12, 13, 14, 15, 16, 17, 18, 19, 20 or 30 amino acids or a number of amino acids between 3 and 30.

Mutants may comprise at least one modification (e.g. addition, substitution, inversion and/or deletion) compared to the corresponding wild-type polypeptide. The mutant may display an altered activity or property, e.g. binding.

Mutations may occur in any of the biomarker proteins and components containing such fragments may serve the purpose of modulating the activity of the mutant to restore, completely or partially the activity of the wild-type polypeptide.

Derivatives may also comprise natural variations or polymorphisms which may exist between individuals or between members of a family. All such derivatives are included within the scope of the invention.

Purely as examples, conservative replacements which may be found in such polymorphisms may be between amino acids within the following groups:

alanine, serine, threonine;

glutamic acid and aspartic acid;

arginine and leucine;

asparagine and glutamine;

isoleucine, leucine and valine;

phenylalanine, tyrosine and tryptophan.

In this specification, a biomarker may be any peptide, polypeptide or protein having an amino acid sequence having a specified degree of sequence identity to one of the biomarker sequences, or to a fragment of one of these sequences. The specified degree of sequence identity may be from at least 60% to 100% sequence identity. More preferably, the specified degree of sequence identity may be one of at least 65%, 70%, 75%, 80%, 85%, 86%, 87%, 88%, 89%, 90%, 91%, 92%, 93%, 94%, 95%, 96%, 97%, 98% or 99% identity.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### EXAMPLES

### Example 1

CVF (cervicovaginal fluid) samples were collected from pregnant women at 19-37 weeks of gestational age. A sterile bivalve speculum was inserted into patients' vagina. A dual-tipped swab was placed in the posterior fornix of the vagina for 30 seconds and then placed into 1 mL of chilled CVF extraction buffer (50 mM HEPES, 150 mM NaCl, 0.1% SDS, 1 mM EDTA, 1 mM Pefabloc SC 4-(2-aminoethyl)benzene sulfonyl fluoride (AEBSF)). Samples were vortexed for 10 s, after which the swab was inverted and was centrifuged for 5 min at 1000×g. The swab was discarded, and the sample tube was vortexed for 10 s before centrifugation at 1000×g for 5 min. The extracted CVF (supernatant) was aliquoted into tubes and stored at −80° C. until required.

Seven protein biomarkers were tested in the 200 CVF samples which were obtained from 86 patients who eventually had term and preterm deliveries (ECM1, GGH, LAMC2, EFEMP1, PTN, FGA and PEDF). The samples were collected longitudinally from 19-38 weeks of gestation. The biomarker expression level in the CVF samples were measured using commercial ELISA kits, namely, PEDF (DuoSet, # DY1177-05, R&D Systems, Minneapolis, Minn.), ECM1 (# ELH-ECM1-1, Raybiotech), GGH (# EH4206, Wuhan Fine Biotech), LAMC2 (# SEC083Hu, Cloud-clone), PTN (#23437, LSBIO), FGA (#11466, LSBIO), EFEMP1 (# MBS178533, MyBioSource). Samples were run as duplicates in a standard 96-well plate alongside a reference control and a standard protein at known concentration.

ELISA protocols were assayed based on the manufacturers manual. Below described the general protocol:

Coat 96 wells with 100 μl Capture Antibody, at a concentration of between 0.8-10 μg/ml in coating buffer. Cover the plate and incubate overnight at 4° C.

Add 300 μl of blocking solution to each well. Incubate for 60 minutes. Wash plate three times with wash buffer and dry by tapping inverted plate on dry paper.

Add 100 μl of Standard protein in serial dilutions and properly diluted samples. Samples or standards are run in duplicates and incubated for 90 min at 37° C. Wash plate three times with wash buffer and dry by tapping inverted plate on dry paper.

Add 100 μl of biotin-conjugated detection antibody, diluted in reagent diluent or appropriate buffer and incubate for 1 hour at 37° C. Wash plate three times with wash buffer.

Add 100 μl of enzyme-conjugated streptavidin, diluted in reagent diluent or appropriate buffer and incubate for 60 minutes at 37° C. Wash plate three times with wash buffer and dry by tapping inverted plate on dry paper.

Add 100 μl of the appropriate substrate solution to each well. Incubate at 37° C. for up to 20 minutes, or until desired colour change is attained.

Read absorbance values immediately at the appropriate wavelength or add 50 μl of “stop solution”. Gently tap plate to ensure thorough mixing. Measure absorbance at 450 nm and referenced at 540 nm.

The biomarker concentration was determined based on the standard curve run on every plate as either a linear or 4 Parameter Logistic (4PL) standard curve. The final concentration was normalized based on the total protein concentration determined by bicinchoninic acid assay (BCA assay).

The values and data of samples were then assembled to compare the Term and Preterm results stratifying based on 3 principal methods:
Entire group in which the entire cohort (n=200) of Term (n=136) and Preterm (n=64) were assessed for difference in mean and demonstrating p-values obtained from Student's t-test analysis.
Gestational week—samples grouped based on the gestational week at sampling
Time from delivery—Samples were grouped based on the number of days between the sample and delivery

### Statistical Analysis.

For statistical analysis, two-tailed unpaired Student's t-test was performed at confidence interval (CI)=0.95 using the Microsoft Excel software, with p-value (P) of less than 0.05 considered significant. All numerical data including error bars represent the mean+/−Standard error of mean (SEM).

### Results and Discussion.

The biomarker quantification on the 200 clinically-derived samples demonstrated a difference between term and preterm samples. Further stratification of the samples enabled the emphasis for potential time points in gestation that will enable a better understanding of preterm birth risk base.

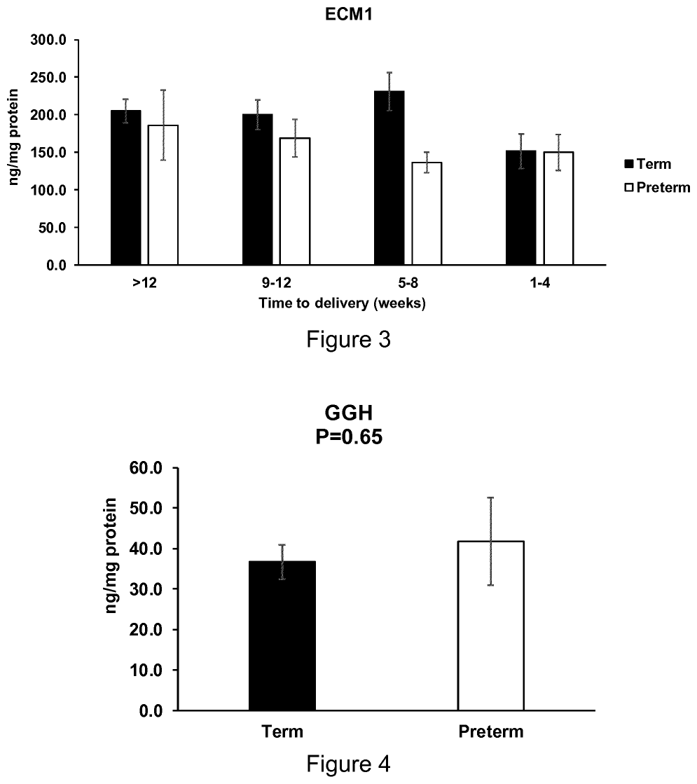

ECM1 was differentially expressed between all 200 term and preterm samples collected with a p-Value of P=0.0025 (FIG. 1). Upon stratification of the samples into different gestational ages and different time from sampling to delivery, the differential expression remained in the same direction (i.e. ECM1 was expressed less on average in all preterm samples regardless of gestational age and time to delivery) (FIG. 2 and FIG. 3). As ECM1 is a known marker in several skin-related disorder and angiogenesis, it is therefore somewhat surprising for its correlation with preterm birth.

GGH was differentially expressed between term and preterm samples. The expression of GGH was on average elevated in samples from preterm women vs samples from term women (FIG. 4). Interestingly, in both term and preterm cases, there was an incremental increase in expression levels as gestational age progressed and time to delivery declined (FIGS. 5 and 6). GGH is not a widely known biomarker, it is involved in immune pathways and extracellular matrix regulation.

LAMC2 was differentially expressed between all 200 term and preterm samples (FIG. 7). In contrast to the other markers, the difference was more pronounced towards the last days before delivery (FIG. 9). LAMC2 is involved in epithelial transition pathways and is known for its involvement in several skin disease indications. Intriguingly, it has never been associated with changes in cervical vaginal space.

EFEMP1 was also differentially expressed between all 200 term and preterm samples (FIG. 10). However, more interestingly there was a transition between elevated expression and reduced expression of EFEMP1 throughout gestation between term and preterm samples. In the early timepoints of both gestational age and time to delivery, EFEMP1 term samples were elevated in comparison to the preterm samples. However, this trend was reversed in later timepoints of gestational ages and time to delivery (FIG. 11 and FIG. 12).

PTN was differentially expressed between all 200 term and preterm samples (FIG. 13). The most pronounced difference in the expression profile of PTN between term and preterm samples was observed in the earliest gestational ages, and in the earliest as well as the latest timepoints in time to delivery (FIGS. 14 and 15).

FGA was differentially expressed between all 200 term and preterm samples (FIG. 16). Differences in FGA expression was more pronounced in late stages of gestation and towards the last days before delivery (FIGS. 17 and 18). FGA as a protein is involved in inflammation and immune response pathways and thus might relate to preterm birth cases initiated by such pathways.

PEDF was differentially expressed between all 200 term and preterm samples (FIG. 19). PEDF was consistently elevated in the preterm samples in all stratifications (FIGS. 20 and 21), indicating that this would be a robust biomarker at any time point. PEDF is a protein tightly related to angiogenesis and thus remodelling of tissue. We hypothesize that its involvement in preterm birth is related to cervical remodelling.

The current state of predicting women at risk for preterm birth is quite limited. The two most common ways to achieve a risk profile are based on prior history and cervical length. These methods fail to correctly assess the risk of preterm birth in the majority of women even when used in combination. Thus, a tool that would accurately predict women at risk of preterm birth would be a great asset to the clinical community in managing pregnancies, and would further allow the reduction of preterm birth cases and saving on the significant health care costs. Here, we demonstrate the corner stones for such a tool via individual biomarkers. As we see it, these biomarkers, ECM1, FGA, EFEMP1, GGH, PEDF, PTN and LAMC2 lay the foundation for a kit that would combine the predictive values of the individual biomarkers, generating a highly accurate tool.

### Example 2: In Vitro Assays

The ectocervical Ect1/E6E7 (ATCC CRL-2614) and endocervical End1/E6E7 (ATCC CRL-2615) cell lines were selected for in vitro study of biomarkers under various cellular stress conditions (Hydrogen peroxide and LPS) based on the follow protocols:

H₂O₂ Treatment

Ect1 and End1 cells were seeded in Keratinocyte serum-free medium (KSFM) supplemented with 0.1 ng/ml EGF, 50 μg/ml BPE and 0.4 mM CaCl₂) at Day 0. Upon reaching 70-80% confluency at day 2, cells were treated with increasing doses of H₂O₂ (50 μM, 100 μM, 200 μM, 400 μM). After 24 hours, culture media was collected for biomarkers quantification using ELISA. Effect of H₂O₂ on cell viability and proliferation was assessed using the MTT assay.

### LPS Treatment

Ect1 and End1 cells were seeded in KSFM supplemented with 0.1 ng/ml EGF, 50 μg/ml BPE and 0.4 mM CaCl₂) at Day 0. At Day 1, cell culture was removed and substituted with KSFM without growth factor supplement. Cells were then treated with increasing doses of LPS (10 μg/ml, 25 μg/ml, 50 μg/ml) at Day 2. At 24 h post-LPS treatment, culture media was collected for biomarkers quantification using ELISA.

### Results and Discussion:

The ectocervical Ect1/E6E7 (ATCC CRL-2614) and endocervical End1/E6E7 (ATCC CRL-2615) cell lines, both derived from normal cervical epithelial tissue, was selected for in vitro study of biomarkers. End1 expresses characteristics of simple columnar epithelium, whereas Ect1 express characteristics of stratified squamous nonkeratinizing epithelia. As CVF is a mixture of fluids originating from the vagina, cervix and adjacent overlying foetal membranes, studying the secreted contents of Ect1 and End1 in the presence of different extracellular stress thus provides references and inferences to the local biochemical milieu and the physiological changes of the cervix during pregnancy.

Oxidative stress has been reported to play important roles in normal term and spontaneous preterm birth (PTB) as well as in many pregnancy complications such as Preterm premature rupture of membranes (PPROM) and pre-eclampsia. Although the molecular mechanisms remain unclear, the roles of oxidative stress in PTB could be attributed to various reactive oxygen species (ROS)-mediated pathophysiological pathways such as inflammation, apoptosis, autophagy, senescence, and altered collagen metabolism. Study by Menon et al showed that in foetal membranes from PTB and PPROM, the expression of oxidative stress markers such as F2-Isoprostanes and OS-induced 3-nitrotyrosine modified proteins (3-NT) was significantly higher than that in foetal membrane from term birth. At the same time, both PTB and PPROM had higher amniotic fluid F2-Isoprostanes than term birth. The same group of researchers further deduced that oxidative stress-mediated apoptosis resulted in proteolysis in foetal membrane that eventually leads to membrane weakening and rupture in PPROM. On the other hand, Heng et at showed that expression of antioxidant enzymes, thioredoxin and SOD1 as well as the total antioxidant capacity of CVF decreased significantly with approaching labour. They concluded that labour is associated with increased oxidative stress and the antioxidant enzymes could potentially serve as predictors of labour.

To induce oxidative stress in our system, Ect1 and End1 cells were treated with increasing doses of H₂O₂ for 24 h. Cell viability and proliferation of H2O2-treated cells were assessed using the MTT assay (FIG. 22). We found that H₂O₂ treatment induced differential cell response in Ect1 and End1 cells. In Ect1 cells, lower dosage of H₂O₂ (50 μM and 100 μM) did not affect cell viability. At 200 μM H2O2, there was a 20% decrease in cell density in treated cells, as compared to control untreated cells, although the decrease is not statistically significant. At 400 μM H2O2, a significant decrease of ˜50% in cell viability in treated cells was observed. In End1 cells, while 50 μM had no effect, 100 μM, 200 μM and 400 μM H₂O₂ resulted in significant decrease of ˜20%, ˜50% and ˜90% in cell viability, respectively.

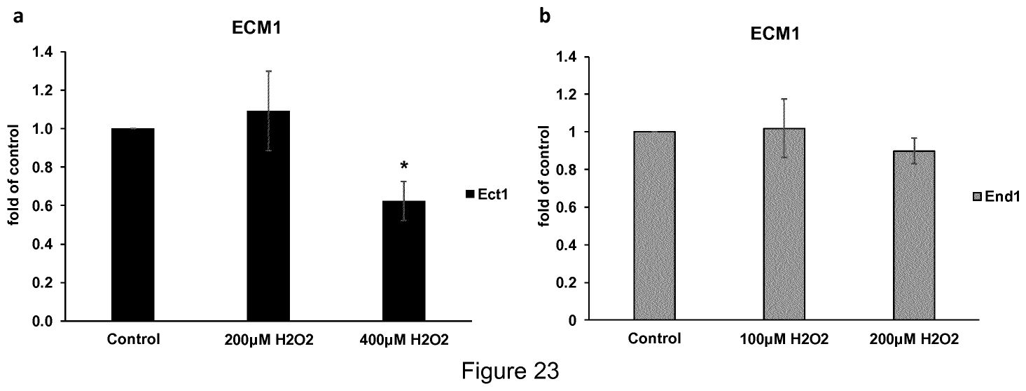

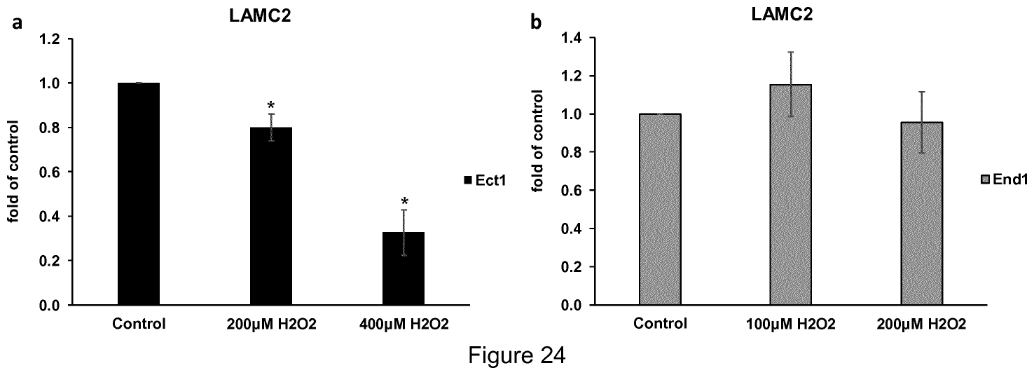

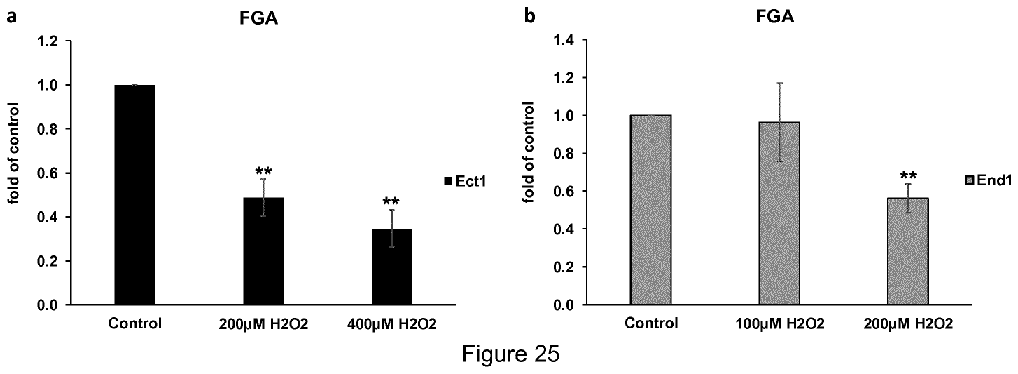

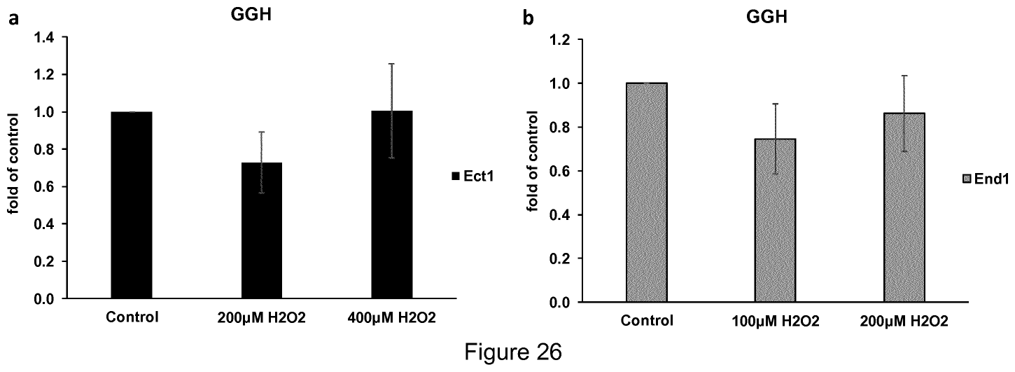

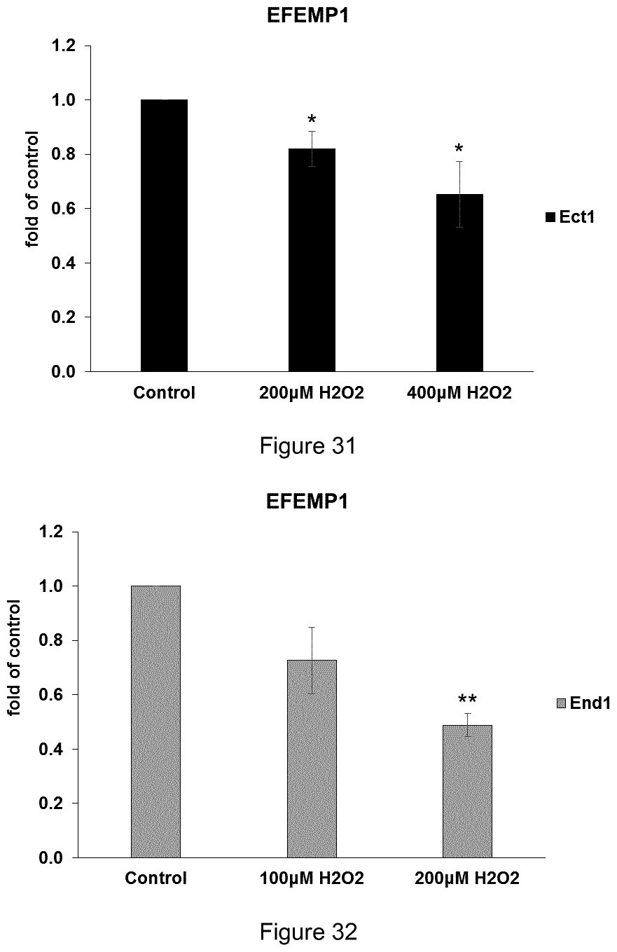

We proceeded with quantifying biomarkers expression in the culture media of cells treated with H₂O₂ using ELISA. Interestingly, the biomarkers showed differential expression upon H₂O₂ treatment. In Ect1, expression of FGA (FIG. 25), LAMC2 (FIG. 24) and EFEMP1 (FIG. 31) in Ect1 was found to decrease significantly upon treatment with 200 μM and 400 μM of H2O2. ECM1 expression decreased (P<0.05) when Ect1 was exposed to 400 μM H₂O₂ for 24 h (FIG. 23). GGH expression remained relatively unchanged upon H₂O₂ treatment (FIG. 26).

For End1 cells, significant change in expression was observed in FGA and EFEMP1 in cells treated with 200 μM of H₂O₂ (FIGS. 25 and 32). The expression of ECM1 (FIG. 23), GGH (FIG. 26) and LAMC2 (FIG. 24) were similar with control untreated cells despite treatment with H₂O₂ for 24 h. Table 1 and table 2 summarize the changes in expression of the biomarkers in Ect1 and End1 upon H₂O₂ treatment.

TABLE 1
Expression of biomarkers in Ect1 following H₂O₂ treatment for 24 hours
Ect1

| Biomarker | Fold change (200 μM H2O2) | Fold change (400 μM H2O2) |
|---|---|---|
| ECM1 | 1.09 ± 0.21 | 0.62* ± 0.10 |
| GGH | 0.73 ± 0.16 | 1.01 ± 0.25 |
| FGA | 0.49** ± 0.08 | 0.35** ± 0.08 |
| LAMC2 | 0.80* ± 0.06 | 0.33* ± 0.10 |
| EFEMP1 | 0.82* ± 0.07 | 0.65* ± 0.12 |

Fold change compared to control untreated cells represents the average of at least 4 independent experiments.
Results are shown as fold change ± SEM.
*P < 0.05,
**P < 0.005.

TABLE 2
Expression of biomarkers in End1 following H₂O₂ treatment for 24 hours
End1

| Biomarker | Fold change (100 μM H2O2) | Fold change (200 μM H2O2) |
|---|---|---|
| ECM1 | 1.02 ± 0.16 | 0.90 ± 0.07 |
| GGH | 0.75 ± 0.16 | 0.86 ± 0.18 |
| FGA | 0.96 ± 0.21 | 0.56** ± 0.08 |
| LAMC2 | 1.15 ± 0.17 | 0.95 ± 0.16 |
| EFEMP1 | 0.73 ± 0.12 | 0.49** ± 0.04 |

Fold change compared to control untreated cells represents the average of at least 3 independent experiments.
Results are shown as fold change ± SEM.
**P < 0.005.

Similar with oxidative stress, inflammation has long been associated with labour and delivery at term and preterm. In most cases, PTB and PPROMs are closely related to intra-amniotic infection, intrauterine infection, and inflammation. Numerous experimental and clinical studies pin-point to pathophysiological pathways mediated by inflammatory mediators as the underlying causes for preterm delivery and several pregnancy complications. Such inflammation-mediated pathways include leukocyte activation, increased inflammatory cytokines and chemokines, and collagenolysis by the extracellular matrix metalloproteinases (MMPs). These events eventually result in loss of membrane structural integrity, myometrial activation, and early/premature cervical remodelling, leading to PTB and PPROM. Furthermore, markers of inflammation, such as the interleukins (IL)1,2,6, and 8, tumour necrosis factor-[TNF-], and C-reactive protein [CRP]) have been evaluated as biomarkers in PTB.

In our study, we induced inflammation in our system by treating Ect1 and End1 cells with various doses of Lipopolysaccharide (LPS).

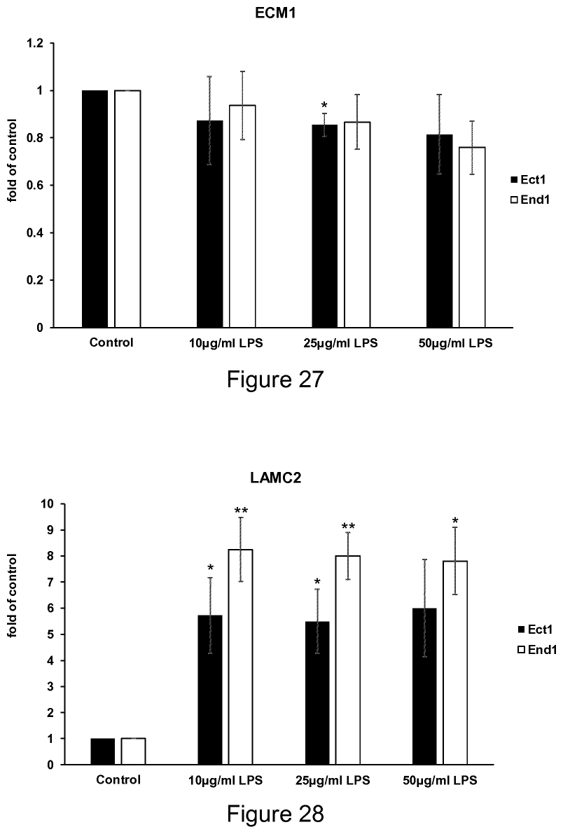

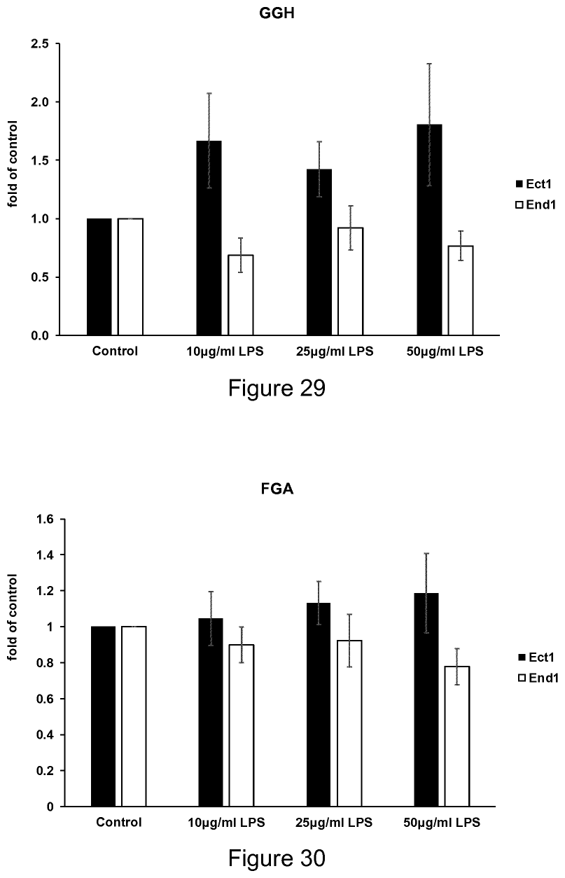

In Ect1 cells, 25 μg/ml LPS treatment resulted in a significant decrease in ECM1 expression in the conditioned media (FIG. 27). Moreover, lower doses of LPS (10 μg/ml and 25 μg/ml) caused statistically-significant increment in LAMC2 expression of more than 5-fold, when compared to control untreated cells (FIG. 28). We also observed slight increases in GGH (FIG. 29) and FGA (FIG. 30) expression in the conditioned media of Ect1 cells treated with 3 different doses of LPS, however, such increase was statistically insignificant.

In End1 cells, similar with Ect1 cells, LPS treatment resulted in a small decrease in ECM1 expression in the conditioned media (FIG. 27), even though such decrease was deemed as statistically insignificant. All three doses of high and low LPS (10 μg/ml, 25 μg/ml, 50 μg/ml) led to ˜8-fold increase in LAMC2 expression in End1 conditioned media (FIG. 28). On the contrary, expression level of GGH (FIG. 29) and FGA (FIG. 30) in End1 conditioned media were down-regulated with LPS treatment. Table 3 and 4 summarize the changes in expression of the biomarkers upon LPS treatment.

TABLE 3
Expression of biomarkers in Ect1 following LPS treatment for 24 hours
Ect1

| | Fold change | Fold change | Fold change |
|---|---|---|---|
| Biomarker | (10 μg/ml LPS) | (25 μg/ml LPS) | (50 μg/ml LPS) |
| ECM1 | 0.87 ± 0.18 | 0.86* ± 0.05 | 0.81 ± 0.17 |
| GGH | 1.67 ± 0.40 | 1.42 ± 0.23 | 1.80 ± 0.52 |
| FGA | 1.05 ± 0.15 | 1.13 ± 0.12 | 1.19 ± 0.22 |
| LAMC2 | 5.72* ± 1.44 | 5.50* ± 1.23 | 5.99 ± 1.86 |

Fold change compared to control untreated cells represents the average of at least 4 independent experiments.
Results are shown as fold change ± SEM.
*P < 0.05.

TABLE 4
Expression of biomarkers in End1 following LPS treatment for 24 hours
End1

| | Fold change | Fold change | Fold change |
|---|---|---|---|
| Biomarker | (10 μg/ml LPS) | (25 μg/ml LPS) | (50 μg/ml LPS) |
| ECM1 | 0.94 ± 0.14 | 0.87 ± 0.11 | 0.76 ± 0.11 |
| GGH | 0.69 ± 0.15 | 0.92 ± 0.19 | 0.76 ± 0.13 |
| FGA | 0.90 ± 0.10 | 0.92 ± 0.15 | 0.78 ± 0.10 |
| LAMC2 | 8.25** ± 1.22 | 8.01** ± 0.90 | 7.81* ± 1.28 |

Fold change compared to control untreated cells represents the average of at least 4 independent experiments.
Results are shown as fold change ± SEM.
*P < 0.05,
**P < 0.005.

### REFERENCES

A number of publications are cited above in order to fully describe and disclose the invention and the state of the art to which the invention pertains. Full citations for these references are provided below. The entirety of each of these references is incorporated herein.
1. Blencowe H, Cousens S, Oestergaard M Z, Chou D, Moller A B, Narwal R, Adler A, Vera Garcia C, Rohde S, Say L, Lawn J E: National, regional, and worldwide estimates of preterm birth rates in the year 2010 with time trends since 1990 for selected countries: a systematic analysis and implications. Lancet, 379(9832):2162-2172.
2. Goldenberg R L, Culhane J F, Iams J D, Romero R: Epidemiology and causes of preterm birth. Lancet, 371(9606):75-84.
3. Stoll B J, Hansen N I, Bell E F, Shankaran S, Laptook A R, Walsh M C, Hale E C, Newman N S, Schibler K, Carlo W A, Kennedy K A, Poindexter B B, Finer N N, Ehrenkranz R A, Duara S, Sánchez P J, O'Shea T M, Goldberg R N, Van Meurs K P, Faix R G, Phelps D L, Frantz I D, Watterberg K L, Saha S, Das A, Higgins R D: Neonatal Outcomes of Extremely Preterm Infants From the NICHD Neonatal Research Network. Pediatrics 2010, 126(3):443-456.
4. Matthews T J, MacDorman M F, Thoma M E: Infant Mortality Statistics From the 2013 Period Linked Birth/Infant Death Data Set. Natl Vital Stat Rep 2015, 64(9):1-30.
5. Blencowe H, Cousens S, Oestergaard M, Chou D, Moller A: National, regional and worldwide estimates of preterm birth rates in the year with time trends for selected countries since a systematic analysis. The Lancet in press 2010, 1990
6. St John E B, Nelson K G, Cliver S P, Bishnoi R R, Goldenberg R L: Cost of neonatal care according to gestational age at birth and survival status. Am J Obstet Gynecol 2000, 182(1):170-175.
7. 1Petrou S: The economic consequences of preterm birth during the first 10 years of life. Lancet 2005, 112:10-15.
8. Gilbert W: The cost of preterm birth: the low cost versus high value of tocolysis. Lancet 2006, 113:4-9.
9. Hamilton B, Martin J, Ventura S J: Births: Preliminary data for 2011. Natl Vital Stat Rep 61(5)
10. Boardman J P: Preterm Birth: Causes, Consequences and Prevention. Lancet 2008, 28(5):559-559.
11. Goldenberg R L, Iams J D, Mercer B M, Meis P J, Moawad A, Das A, Miodovnik M, VanDorsten P J, Caritis S N, Thurnau G, Dombrowski M P: The Preterm Prediction Study: Toward a multiple-marker test for spontaneous preterm birth. Am J Obstet Gynecol 2001, 185(3):643-651.
12. Koullali B, Oudijk M, Nijman T, Mol B, Pajkrt E: Risk assessment and management to prevent preterm birth. Lancet 2016, 21(2):80-88.
13. Slattery M M, Morrison J J: Preterm delivery. Lancet 360(9344):1489-1497.
14. Schaaf J M, Liem S M, Mol B W J, Abu-Hanna A, Ravelli A C: Ethnic and Racial Disparities in the Risk of Preterm Birth: A Systematic Review and Meta-Analysis. Lancet 2013, 30(06):433-450.
15. Ananth C V, Vintzileos A M: Epidemiology of preterm birth and its clinical subtypes. Lancet 2006, 19(12):773-782.
16. Cnattingius S, Villamor E, Johansson S, et al: Maternal obesity and risk of preterm delivery. JAMA 2013, 309(22):2362-2370.
17. Murphy D J: Epidemiology and environmental factors in preterm labour. Lancet 2007, 21(5):773-789.
18. HASSAN S S, ROMERO R, VIDYADHARI D, FUSEY S, BAXTER J K, KHANDELWAL M, VIJAYARAGHAVAN J, TRIVEDI Y, SOMA-PILLAY P, SAMBAREY P, DAYAL A, POTAPOV V, O′BRIEN J, ASTAKHOV V, YUZKO O, KINZLER W, DATTEL B, SEHDEV H, MAZHEIKA L, MANCHULENKO D, GERVASI M T, SULLIVAN L, CONDE-AGUDELO A, PHILLIPS J A, CREASY G W: Vaginal progesterone reduces the rate of preterm birth in women with a sonographic short cervix: a multicenter, randomized, double-blind, placebo-controlled trial. Ultrasound Obstet Gynecol 2011, 38(1):18-31.
19. Meis P J, Klebanoff M, Thom E, Dombrowski M P, Sibai B, Moawad A H, Spong C Y, Hauth J C, Miodovnik M, Varner M W, Leveno K J, Caritis S N, Iams J D, Wapner R J, Conway D, O'Sullivan M J, Carpenter M, Mercer B, Ramin S M, Thorp J M, Peaceman A M: Prevention of Recurrent Preterm Delivery by 17 Alpha-Hydroxyprogesterone Caproate. N Engl J Med 2003, 348(24):2379-2385.
20. American College of Obstetricians and Gynecologists: Use of progesterone to reduce preterm birth: ACOG committee opinion no. 419. Obstet Gynecol 2008, 112:963-965.
21. Suhag A, Berghella V: Cervical cerclage. Clin Obstet Gynecol 2014, 57(3):557-567.
22. OWEN J, HANKINS G, IAMS J D, BERGHELLA V, SHEFFIELD J S, PEREZ-DELBOY A, EGERMAN R S, WING D A, TOMLINSON M, SILVER R, RAMIN S M, GUZMAN E R, GORDON M, HOW H Y, KNUDTSON E J, SZYCHOWSKI J M, CLIVER S, HAUTH J C: Multicenter randomized trial of cerclage for preterm birth prevention in high-risk women with shortened midtrimester cervical length. Am J Obstet Gynecol 2009, 201(4):375.e1-375.e8.
23. Berghella V, Rafael T J, Szychowski J M, Rust O A O J: Berghella V, Rafael T J, Szychowski J M, Rust O A, Owen J. Cerclage for short cervix on ultrasonography in women with singleton gestations and previous preterm birth: a meta-analysis. Obstet Gynecol 2011, 117(3):663-671.
24. Heng Y J, Liong S, Permezel M, Rice G E, Di Quinzio M K W, Georgiou H M: Human cervicovaginal fluid biomarkers to predict term and preterm labor. Front Physiol 2015, 6:151.
25. Dunietz G L, Holzman C, McKane P, Li C, Boulet S L, Todem D, Kissin D M, Copeland G, Bernson D, Sappenfield W M, Diamond M P: Assisted reproductive technology and the risk of preterm birth among primiparas. Fertil Steril 2015, 103(4):974-979.e1.

For standard molecular biology techniques, see Sambrook, J., Russel, D. W. Molecular Cloning, A Laboratory Manual. 3 ed. 2001, Cold Spring Harbor, N.Y.: Cold Spring Harbor Laboratory Press

1. A method for predicting whether an individual is at risk of preterm birth, the method comprising determining the level of a biomarker in a sample obtained from the individual, and predicting whether the individual is at risk of preterm birth based on the level of the biomarker, wherein the biomarker is selected from ECM1, FGA, EFEMP1, GGH, PEDF, PTN and LAMC2.

2. The method of claim 1, wherein the sample is a sample of vaginal fluid.

3. The method of claim 1 or claim 2, wherein the biomarker is a protein.

4. The method of any one of claims 1 to 3, wherein the level of a biomarker is compared to a reference level, wherein the reference level is derived from the level of biomarker in a sample obtained from an individual known to have experienced preterm or term birth.

5. The method according to any one of claims 1 to 4, wherein the method further comprises predicting the risk of preterm birth with one or more other indicators of preterm birth, selected from the Fetal Fibronectin (fFN) test, contractions, vaginal bleeding, fluid leaking from the vagina, increased vaginal discharge, backache and cramping in lower abdomen.

6. Progesterone for use in the treatment of an individual predicted to be at risk of preterm birth, wherein the individual has been predicted to be at risk of preterm birth by a method according to any one of claims 1-4.

7. A method for selecting an individual for treatment to reduce the risk of preterm birth, the method comprising predicting the risk of preterm birth in the individual using a method according to any one of claims 1 to 4 and, if the individual is determined to be at risk of preterm birth, administering a treatment to reduce the risk of preterm birth, wherein the treatment to reduce the risk of preterm birth comprises progesterone and/or cervical cerclage.

8. A method for predicting whether an individual is at risk of preterm birth, the method comprising determining the level of a biomarker in a sample obtained from the individual, and transmitting the determined level to a physician involved in the treatment of the individual, wherein the risk of preterm birth is predicted, based on the level of the biomarker in the sample, and wherein the biomarker is selected from ECM1, FGA, EFEMP1, GGH, PEDF, PTN and LAMC2.

9. The method according to any one of claims 1 to 8, wherein the method for predicting whether the individual is at risk of preterm birth is a computer implemented method.

10. A method for detecting ECM1, FGA, EFEMP1, GGH, PEDF, PTN or LAMC2, the method comprising:
a. obtaining a sample of vaginal fluid from an individual;
b. detecting whether ECM1, FGA, EFEMP1, GGH, PEDF, PTN or LAMC2 is present in the vaginal fluid sample by contacting the vaginal fluid sample with an anti-ECM1, anti-FGA, anti-EFEMP1, anti-GGH, anti-PEDF, anti-PTN or anti-LAMC2 antibody and detecting binding between ECM1, FGA, EFEMP1, GGH, PEDF, PTN or LAMC2 and the antibody.

11. A method for determining that an individual is at risk of preterm birth, said method comprising:
a. obtaining a sample from an individual;
b. detecting whether ECM1, FGA, EFEMP1, GGH, PEDF, PTN or LAMC2 is present in the sample by contacting the sample with an anti-ECM1, anti-FGA, anti-EFEMP1, anti-GGH, anti-PEDF, anti-PTN or anti-LAMC2 antibody and detecting binding between ECM1, FGA, EFEMP1, GGH, PEDF, PTN or LAMC2 and the antibody; and
c. determining that the individual is at risk of preterm birth when the presence of ECM1, FGA, EFEMP1, GGH, PEDF, PTN or LAMC2 in the sample is detected.

12. The method of claim 11 wherein the sample is a vaginal fluid sample.

13. A method of determining that an individual is at risk of preterm birth and prolonging gestation in that individual, the method comprising:
a. obtaining a sample from an individual;
b. detecting whether ECM1, FGA, EFEMP1, GGH, PEDF, PTN or LAMC2 is present in the vaginal fluid sample;
c. determining that the individual is at risk of preterm birth when the presence of ECM1, FGA, EFEMP1, GGH, PEDF, PTN or LAMC2 in the vaginal fluid sample is detected; and
d. administering an effective amount of progesterone to the individual determined to be at risk of preterm birth or selecting the individual for treatment with an effective amount of one or more agents selected from a progesterone or an analogue thereof, a tocolytic, a corticosteroid, an antibiotic, an NSAID or an Omega 3 fatty acid or derivative thereof if the individual is determined to be at risk of preterm birth; and/or
e. performing cervical cerclage on the individual determined to be at risk of preterm birth or selecting the individual for cervical cerclage, if the individual is determined to be at risk of preterm birth

14. The method according to any one of claims 10 to 13, wherein the level of the biomarker is determined by ELISA.

15. A kit for use in predicting the risk or likelihood of preterm birth in a subject, the kit comprising anti-ECM1, anti-FGA, anti-EFEMP1, anti-GGH, anti-PEDF, anti-PTN or anti-LAMC2 antibody.
